(19) United States
(12) Patent Application Publication
(10) Pub. No.: US 2020/0020426 A1
(43) Pub. Date: Jan. 16, 2020

(54) METHODS AND SYSTEMS FOR STAFFING IN-HOME HEALTHCARE SERVICES

(71) Applicant: VISCARE TECHNOLOGIES, LLC, Grapevine, TX (US)

(72) Inventors: Sheri A. Yarbray, Grapevine, TX (US); Jeffrey D. Moffett, Grapevine, TX (US)

(73) Assignee: VISCARE TECHNOLOGIES, LLC, Grapevine, TX (US)

(21) Appl. No.: 16/508,632

(22) Filed: Jul. 11, 2019

Related U.S. Application Data
(60) Provisional application No. 62/696,496, filed on Jul. 11, 2018.

(51) Int. Cl. G16H 10/60; G16H 20/30; G16H 80/00

(52) U.S. Cl. CPC G16H 10/60; G16H 40/20; G16H 80/00; G16H 20/30

(57) ABSTRACT

In one embodiment, a computer-implemented method includes receiving a referral includes patient data, where the patient data indicates a patient is referred for in-home therapy. The method also includes generating, by a processing device, a group of candidate clinicians from a dataset of clinicians, where the group of candidate clinicians are matches for providing the in-home therapy to the patient, and the generating is based at least on criteria comprising a candidate clinician having the certain discipline, a current utilization rate of the candidate clinician relative to an overall capacity of the candidate clinician to work over a period of time, and a billing rate the candidate clinician charges per visit. The method also includes assigning, by the processing device, one clinician from the group of candidate clinicians to perform the in-home therapy for the patient.

### CROSS-REFERENCE TO RELATED APPLICATIONS

The present application claims the benefit of U.S. Provisional Application No. 62/696,496, titled ‘METHODS AND SYSTEMS THAT PRODUCE BETTER PATIENT OUTCOMES FOR IN-HOME HEALTHCARE SERVICES”filed Jul. 11, 2018, the content of which is incorporated herein by reference in its entirety for all purposes.

### BACKGROUND

The provisioning of in-home health care services (e.g., physical therapy, occupational therapy, speech therapy, skilled in-home nursing) is a very complicated endeavor involving multiple participants and interested parties, well beyond just the patient and clinician. The related-art system has its foundation in a time period before widespread computer use, and before health care information was as statutorily protected as it is today (e.g. Health Insurance Portability and Accountability Act (HIPAA)). The result is that islands of limited automation have created competing and incompatible systems. The overall system is held together largely by reliance on manual data transcription between systems and extensive human oversight. Compliance and utilization factors associated with in-home health care services are well below what is acceptable in other industries.

### SUMMARY

Representative embodiments set forth herein disclose various techniques for staffing in-home healthcare.

In one embodiment, a computer-implemented method includes receiving a referral comprising patient data, where the patient data indicates a patient is referred for in-home therapy. The method also includes generating, by a processing device, a group of candidate clinicians from a dataset of clinicians, where the group of candidate clinicians are matches for providing the in-home therapy to the patient, and the generating is based at least on criteria comprising a candidate clinician having the certain discipline, a current utilization rate of the candidate clinician relative to an overall capacity of the candidate clinician to work over a period of time, and a billing rate the candidate clinician charges per visit. The method also includes assigning, by the processing device, one clinician from the group of candidate clinicians to perform the in-home therapy for the patient.

In some embodiments, a system includes a memory storing instructions and a processor communicatively coupled to the memory to execute the instructions to perform one or more of the operations described above. Further, a tangible, non-transitory computer-readable medium may store instructions that, when executed by a processor, cause the processor to perform one or more of the operations described above.

Other technical features may be readily apparent to one skilled in the art from the following figures, descriptions, and claims.

### BRIEF DESCRIPTION OF THE DRAWINGS

For a detailed description of example embodiments, reference will now be made to the accompanying drawings in which:

FIG. 1 shows, in block diagram form, a high-level system showing the many participants in the provisioning of in-home healthcare services performed by certified healthcare clinicians;

FIG. 2 shows, in block diagram form, an example workflow and underlying system;

FIG. 3 shows, in block diagram form, a system for coordination and control of in-home healthcare services, in accordance with at least some embodiments;

FIG. 4 shows, in block diagram form, an example workflow for generating a group of candidate Clinicians and transmitting notifications to the group of candidate Clinicians, in accordance with at least some embodiments;

FIG. 5 shows, in block diagram form, an example workflow for a Scheduler selecting a candidate Clinician to assign for in-home therapy for a patient, in accordance with at least some embodiments;

FIG. 6 shows, in block diagram form, an example workflow for automatic assignment of a Clinician to provide in-home therapy for a patient, in accordance with at least some embodiments;

FIG. 7 shows an example method for generating a group of candidate Clinicians and assigning a Clinician to perform in-home therapy for a patient, in accordance with at least some embodiments;

FIG. 8 shows an example method for transmitting notifications to a group of candidate Clinicians, in accordance with at least some embodiments;

FIG. 9 shows, in block diagram form, an example workflow for electronic credential management, in accordance with at least some embodiments;

FIG. 10 shows, in block diagram form, an example workflow for providing a notification to a Clinician when a credential is going to expire within a time period, in accordance with at least some embodiments;

FIG. 11 shows, in block diagram form, an example workflow for providing a notification to a Clinician when a credential is expired, in accordance with at least some embodiments;

FIG. 12 shows an example method for electronic credential management, in accordance with at least some embodiments;

FIG. 13 shows an example method for providing a notification to a Clinician when a credential is going to expire within a time period, in accordance with at least some embodiments;

FIG. 14 shows an example method for determining whether requirements pertaining to a credential issued to a Clinician are satisfied, in accordance with at least some embodiments;

FIG. 15 shows, in block diagram form, an example workflow for real-time or near real-time communication between a Physician and a Clinician, in accordance with at least some embodiments;

FIG. 16 shows, in block diagram form, an example workflow for real-time or near real-time communication between a patient and a Clinician, in accordance with at least some embodiments;

FIG. 17 shows, in block diagram form, an example workflow for real-time or near real-time communication between a Scheduler and a Clinician, in accordance with at least some embodiments;

FIG. 18 shows, in block diagram form, an example workflow for creating patient therapy data having an organizational structure of an EMR platform, in accordance with at least some embodiments;

FIG. 19 shows an example method for using a communication mapping to provide real-time or near real-time messaging between computing devices, in accordance with at least some embodiments;

FIG. 20 shows an example method for transmitting encrypted messages with attachments between computing devices, in accordance with at least some embodiments;

FIG. 21 shows an example method for creating patient therapy data having an organizational structure of an EMR platform, in accordance with at least some embodiments;

FIG. 22 shows, in block diagram form, an example workflow for determining a root cause of a therapy session having a favorable outcome and providing a recommendation, in accordance with at least some embodiments;

FIG. 23 shows an example method for determining a root cause of a therapy session having a favorable outcome and providing a recommendation, in accordance with at least some embodiments;

FIG. 24 shows an example method for assigning pay-for-performance metrics, in accordance with at least some embodiments;

FIG. 25 shows an example method for determining whether a satisfactory amount of a type of therapy session are being provided by clinicians, in accordance with at least some embodiments;

FIG. 26 shows an example method for assigning a new Clinician to provide in-home therapy when another Clinician is unavailable, in accordance with at least some embodiments; and

FIG. 27 shows an example computer system in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

A “Clinician” may refer to a therapist, a nurse, or any suitable healthcare personnel that is trained and/or licensed with one or more credentials.

The terms “Doctor” and “Physician” may be used interchangeably herein.

The terms “central services system” and “healthcare platform” may be used interchangeably herein.

The terms “real-time or near real-time” refer to a definite period of time to send and receive a message.

### DETAILED DESCRIPTION

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various embodiments are directed to methods and systems for coordination and control of in-home health care services, such as in-home therapy, nursing, and/or care services. More particularly, various embodiments discussed below address the inability of existing computer systems to coordinate across multiple health care providers (aka physicians and their respective administrative support teams and their respective practice management platforms), Home Health Agencies (and their respective Electronic Medical Record platforms), multiple Staffing Companies (and their respective Electronic Medical Record platforms), and multiple licensed Clinicians. Moreover, the example methods and systems improve the technological field of data analytics regarding in-home health care services, and such data analytics is a missing component in future, but statutorily mandated health care pay-for-performance models of provider reimbursement. The example methods and system also enable the computer systems to provide functionality not possible in related-art systems, including real-time or near real-time doctor involvement in oversight, control, and modification of the patient's therapy and recovery protocols via direct communication with licensed Clinicians treating the patients. The specification first turns to an explanation of related-art systems to highlight the complexity, patient care shortcomings, and inability of existing computer and mobile computing systems to implement suitable coordination and control across the many disparate systems involved in providing patient care.

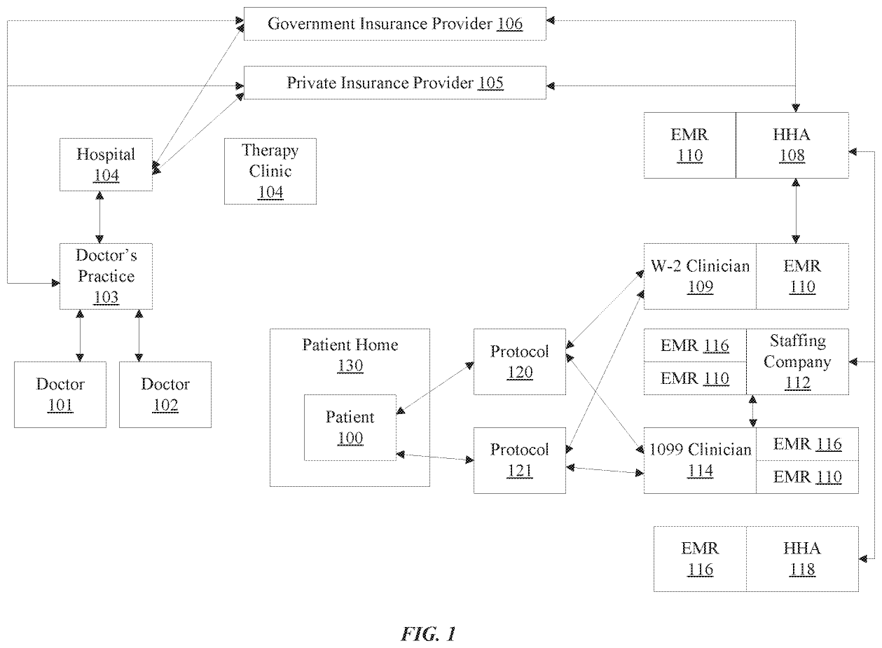

FIG. 1 shows, in block diagram form, a high-level system showing the many participants and variations in provisioning home health care services. For purposes of explanation the discussion assumes a patient (e.g., patient 100) has had an orthopedic surgery, such as a knee replacement, and needs in-home therapy (e.g., physical therapy and occupational therapy) to ensure return of mobility and health. To achieve the desired outcome, the patient receives expedited care by an engaged team of providers with proper oversight to ensure quality. Multiple services are invoked including medication management, wound care, physical therapy, occupational therapy, and other forms of care. The current government regulatory bodies are driving a change in the medical model of reimbursement from fee for service to Value Based Care, where the physician is rewarded for decreasing the amount spent on the patient during post-operative care while at the same time improving outcomes. We are entering a new era of medicine where physicians have potential for increased reimbursement based on performance but do not have adequate tools to ensure cost efficient care. When all parties are engaged in coordinated care made possible by utilizing improved computer and information technology (IT) systems, a more efficient path to a good outcome can be obtained. However, the context of in-home therapy after orthopedic surgeries shall not be read as a limitation. The provisioning of in-home therapy is used in many surgical and non-surgical fields.

For purposes of explanation, assume that the Doctor 101 performs a surgical procedure on the Patient 100 in the Hospital 104 or in a surgical center that is associated with Doctor's Practice Facility 103 (hereafter just Doctor's Practice 103). Both the Doctor 101 and Hospital 104 will ultimately be reimbursed by a Private Insurance Provider 105 (such as Blue Cross Blue Shield (BCBS) or Aetna) or a Government Insurance Provider 106 (such as Medicare or Medicaid) according to established service agreements between the Insurance Provider 105/106), the Doctor 101, and the Doctor Practice 103. Prior to the surgery in this example, the Patient 100 is referred to a Home Health Agency (HHA) 108 either by the Hospital 104 or the Doctor's Practice 103. After the surgery, the Patient 100 is released from the Hospital or out-patient surgery center affiliated with the Doctor's Practice 103 and sent home to recover and receive appropriate therapeutic care from licensed clinicians. Therapeutic care may face certain restrictions based upon insurance provider and Doctor recommendations. For example, Government Insurance Provider 106 may only pay for nine in-home therapeutic sessions, while Private Insurance Provider 105 may pay for 12 in-home therapeutic sessions. By way of further example, a Doctor 101/102 may have his or her own custom physical therapy protocols 120 that she would like Clinician to apply to Patient 100. In other instances, Doctor 101/102 does not have a custom therapy protocol (e.g., for knee replacement surgery), so the Clinician uses a standard therapy protocol 121 as approved and authorized by Insurance Providers 105 or 106 for use with Patient 100 recovery.

The example Doctor 101 and the Doctor's Practice 103 are ultimately responsible for the outcome of Patient's 100 procedure. In some instances, the Doctor 101 determines the Patient 100 should receive therapy at Patient's Home 130 rather than therapy at the Hospital 104 or a separate Therapy Clinic 104. To coordinate the Patient 100 in-home therapy, the Doctor's Practice 103 enters a business relationship with HHA 108, in which HHA 108 coordinates in-home care for Patient 100 via an employee Clinician 109 (e.g. full-time (W-2)) or via an out-sourced (1099) Clinician 114. The HHA 108 is responsible for ensuring the Patient 100 receives in-home therapy according to Protocols 120 or 121, and providing billing and Patient 100 data back to either the Government Insurance Provider 106 or Private Insurance Provider 106. HHA 108 also provides Patient 100 healthcare records to both Insurance Providers 105/106 and to Doctor's Practice 103 for Doctor 101 to review and approve or amend as needed.

There are over 20,000 HHA operations across the United States. Because of HIPAA compliance issues (among others), each HHA utilizes an Electronic Medical Records (EMR) platform. Each EMR platform is one or more computer programs running in any suitable location (e.g., as a Software as a Service (SAAS) system, a computer system physically hosted at the HHA office, or a computer system hosted “off-site” and provided as a cloud-based service). There are dozens of commercially available EMR platforms, in addition to an untold number of proprietary EMR platforms created by individual HHAs for use within their own business. Each EMR platform has its own data structures, communications schemes, subscription access plans, and login credentials. In the example case of HHA 108, the HHA 108 uses EMR platform 110. The Hospital 104 and Insurance Providers 105 and 106 each may have their own EMR platform as well (not specifically shown in FIG. 1).

HHAs may have one or more full-time, W-2 Clinicians 109 on-staff to provide in-home therapy to the Patient 100; however, in some instances, HHAs may elect to concentrate their business efforts on the administrative side (accounting and patient records coordination) of the business. Thus, many HHAs use independent contractor (hereafter 1099 Clinicians 114 reflecting their wages reported by 1099 rather than W2). To provide the in-home therapy, and thus HHAs may enter separate business contracts with Staffing Companies 112 to provide for 1099 Clinician 114 coordination. In the example system of FIG. 1, the example HHA 108 may provide in-home therapy by its own staff W2 Clinician 109, or it may contract with the example Staffing Company 112 to provide 1099 Clinician 114 as needed. Staffing companies source 1099 Clinicians 114 to provide the in-home therapy, and thus the Staffing Companies 112 may have relationships with many 1099 Clinicians 114. Thus, the example Staffing Company 112 may contract with 1099 Clinicians 114 as needed. It is also possible for the Staffing Companies 112 to have their own employee clinicians, but such is not expressly shown in FIG. 1. As mentioned above, each Staffing Company 112 utilizes an EMR platform. Example Staffing Company 112 thus has EMR platform 116.

In most cases, a Clinician (e.g. W2 Clinician 109 or 1099 Clinician 114) is considered to be fully utilized when the Clinician can conduct 30 in-home therapy session equivalents per week. Single therapy sessions takes approximately 45-60 minutes and are counted as a single therapy session, while an initial patient evaluation typically takes 90-120 minutes and is counted as two therapy sessions. In order to achieve the full utilization, many 1099 Clinicians 114 have relationships with multiple Staffing Companies 112 and/or HHAs 108. That is, considering that the Clinician must drive to each Patient's Home 130 to provide in-home therapy session, the 1099 Clinician 114 may have working relationships with multiple Staffing Companies or HHAs to ensure access to a sufficient number of patient therapy sessions to coordinate travel logistics and scheduling that achieves the full utilization and thus maximize revenue potential.

Still referring to FIG. 1, the Patient 100 may thus be referred to HHA 108 by the Doctor's Practice 103. If the HHA 118 elects to provide in-home therapy to Patient 100, then W2 Clinician 109 will provide the in-home therapy according to authorized therapy Protocol 120 or 121 to Patient 100. During such in home therapy sessions, the W2 Clinician 109 will utilize a “thin client” version EMR 110 on a portable computing device (such as a smart phone or tablet) to gather, document, and transmit patient data back to HHA 108. Staff at HHA 108 then conduct quality control measures on the patient data within the EMR 110 prior to reporting that patient data back to the Insurance Providers 105/106 for payment/reimbursement.

The HHA 108 may alternatively, at its discretion, elect to contract with the Staffing Company 112 to provide the in-home therapy sessions to Patient 100. Staffing Company 112 may, in turn, coordinate with its own W2 Clinicians (again not specifically shown in FIG. 1) or a 1099 Clinician 114 to provide the actual in-home therapy Protocol 120/121. For each in-home therapy session, 1099 Clinician 114 gathers, documents, and provides patient data back to the Staffing Company 112 via EMR 110, which is the same EMR utilized by HHA 108. The Staffing Company 112 performs quality control checks on the data within EMR 110, and provides the data back to the HHA 108. The HHA 108, in turn, provides the data back to the Insurance Provider 105/106. However, 1099 Clinician 114 may be contracted with multiple Staffing Companies to provide in home therapy to another patient, for example a patient of HHA 118 rather than HHA 108. In this instance, the HHA 118 utilizes EMR 116 rather than EMR 110, and so the 1099 Clinician 114 gathers, documents, and provides patient data back to the Staffing Company 112 via a “thin client” affiliated with a second EMR 116, via the same portable computing device (such as a smart phone or table) in each in-home therapy session. In so doing, it is easy to see how 1099 Clinician 114 could be easily be confused, or become less efficient, as the Clinician is forced gather, record, and transmit the data from multiple in-home patients across multiple EMRs. The specification now turns to a more detailed workflow for the example situation of provisioning in-home care for the Patient 100.

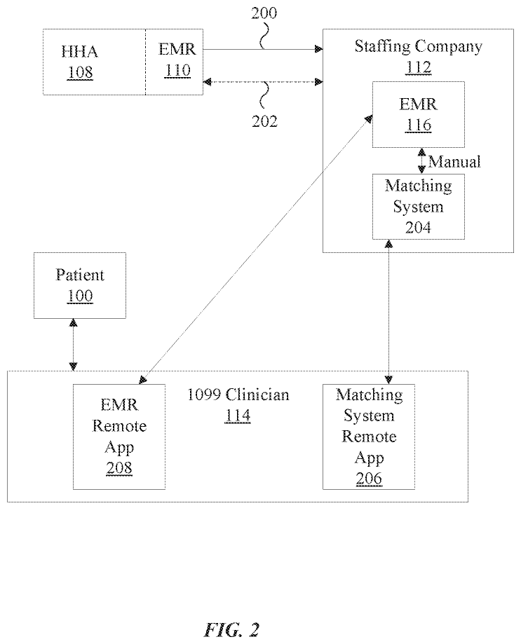

FIG. 2 shows, in block diagram form, an example workflow and underlying system to further highlight the shortcomings of the related-art computer systems and inefficiencies in the related-art systems. In particular, the example HHA 108 may contact the example Staffing Company 112, as shown by arrow 200. Regardless of how the Staffing Company 112 is notified, the Staffing Company 112 then manually logs into the EMR platform 110 of the HHA 108 to retrieve the patient data, as shown by double-arrow dashed line 202. Alternatively, Staffing Company 112 may maintain its own instance and/or license to the same EMR platform (EMR 116) and patient data may be exchanged between EMR 110 and EMR 116. If the Staffing Company 112 works with multiple HHAs, then the Staffing Company 112 may be forced to maintain multiple different versions of EMR 116 at its own expense. Thereafter, the patient data is manually transcribed from the EMR Platform 116 to the matching system 204 of the Staffing Company 112. In years past, the matching system 204 was literally a whiteboard with names of W2 and/or 1099 Clinicians. An employee of the Staffing Company 112 would start contacting each Clinicians on the Staffing Company's roster of qualified clinicians until one was found that matched the patient's requirements and agreed to accept the patient. Some staffing companies recently have automated the matching system 204 in the sense that contact with the 1099 Clinicians may initially be made by electronic mail or text message, but otherwise the process remains similar to the whiteboard-based methods. Regardless of the precise matching technique, the example 1099 Clinicians 114 is identified and agrees to provide in-home therapy to the Patient 100.

An initial in-home therapy session between a 1099 Clinician 114 and the Patient 100 may be of extended length for a variety of reasons. Nevertheless, once the in-home therapy session is complete the 1099 Clinician 114 must perform several non-therapy tasks. First, the 1099 Clinician 114 acquires a signature of the Patient 100 or other responsible person evidencing that the in-home therapy session was actually performed. In years past, the signature was acquired exclusively on paper. Some, but not all, providers now acquire signatures electronically, such as by way of a matching system remote application 206 or via EMR Remote App 208 run on a portable computer system (e.g., smart phone, or tablet device). The matching system remote application 206 communicates with the matching system 204 of the Staffing Company 112 to transfer data (such as the signature). Additionally, the Clinician 114 may log into the EMR Platform 116 of the Staffing Company 112 or the EMR Platform 110 of the HHA 108 to provide patient therapy data (e.g., range of motion, state of the incision, etc.). That is, using an EMR remote application 208, the Clinician may log into the EMR platform 116 of the Staffing Company 112 or into the EMR platform 110 of the HHA 108 to provide the patient therapy data. The EMR remote application 208 may run on a portable computer system (e.g., smart phone or tablet device), and if the 1099 Clinician 114 is lucky, the portable computer system used is the same as the portable computer system hosting the matching system remote application 206. In other cases, the Clinician may have multiple portable computer systems, one for each matching system 206.

If the in-home therapy session is an initial visit, the 1099 Clinician 114 will also attempt to schedule follow-up in-home therapy sessions. In the example case of a knee replacement, the best therapy will initially involve several consecutive days of in-home therapy sessions, and thus next-day scheduling is not uncommon. However, for reasons discussed more below, a majority of next-day visits for in-home therapy sessions are missed because approval from the HHA 108 and/or Insurance Provider 105 or 106 (FIG. 1) cannot propagate through the system quickly enough. A few additional considerations regarding the 1099 Clinician 114. As noted with respect to FIG. 1, 1099 Clinician 114 may do contract work for multiple Staffing Companies in addition to Staffing Company 112. The 1099 Clinician may also do contract work for multiple Home Health Agencies in addition to HHA 108. Each Staffing Company and HHA may have its own EMR platform with its own login credentials. Each Staffing Company and HHA may have its own matching system and corresponding matching system remote application. Thus, a single 1099 Clinician 114 may have dozens of credentials and EMR Remote Apps to keep track of and use each day, and may have multiple matching systems 204 to interface with, all in an attempt to increase income by trying to reach full utilization of 30 in-home therapy sessions a week. Next, the example Clinician 114 is at least three layers removed from the Doctor 101 that performed the example surgery. If the Patient 100 has complications (e.g., infected incision), unless the 1099 Clinician 114 can reach the Doctor 101 to ensure a follow-up appointment is immediately scheduled, the 1099 Clinician 114 may have no choice but to call 911 emergency services regarding those complications, even if a complication is otherwise not a full emergency medical situation. Even if the 1099 Clinician 114 can reach the Doctor 101 (highly unlikely in today's environment), the Doctor likely has yet to receive or review any patient therapy data, and the Doctor has no visualization (e.g., pictures) of the issues found by the 1099 Clinician 114. These and related issues are addressed, at least in part, by the example embodiments discussed below.

Returning to the example workflow of FIG. 2. Once the patient therapy data is in the EMR platform 116 of the Staffing Company 112, or the EMR platform 110 of HHA 108, quality assurance checks are manually run on the data. That is, a human looks through each set of patient therapy data for an in-home therapy session (sometimes referred to as a “patient note” or “note”) to ensure that all the required fields are filled out and that no obvious errors are present. In spite of this quality assurance check, missing fields and incorrect data are overlooked regularly in the human-based analysis. The patient therapy data may wait in a queue for a day or more before being subjected to quality assurance checks, and if errors are found the quality control process starts anew. If HHA 108 contracted with Staffing Company 112, then once checked the patient therapy data may then be transferred from the EMR platform 116 of the Staffing Company 112 to the EMR platform 110 of the HHA 108. The patient data within the EMR platform 110 may again be subjected to quality assurance checks by the HHA 108, which may or may not need to further pass the data along to the Insurance Providers 105/106 (FIG. 1). Eventually however, and assuming no errors are found (which effectively resets the process), the HHA 108 approves billing for the visit and/or approves the schedule and number of follow-up visits. However, the data flow, quality assurance, and approval process may take days. And as mentioned above, the process results in missed in-home therapy sessions a majority of the time when the 1099 Clinician 114 is attempting to schedule a next-day session early in the therapy process. What is more, the Doctor 101 (FIG. 1) is unlikely to have access to any of the patient therapy data until after the prescribed number of in-home therapy sessions (the number usually dictated by Insurance Provider 105/106) is complete.

As shown above, the process is highly inefficient. The inefficiency is driven in large part by an inability of the underlying computer systems to communicate with each other. The limitations result in a reduced amount of data movement (to reduce the manually transcription time and effort) and thus limit data analytics that could otherwise be run and used to improve the process. Relatedly, government-based Insurance Providers 106 (FIG. 1) are moving toward a pay-for-performance system, where quality of patient outcomes dictate reimbursement rates, with poor patient outcomes from lower quality doctors, hospitals, and/or Clinicians making less, and better patient outcomes from higher quality doctors, hospitals, and/or Clinicians making more. The pay-for-performance model will dictate better visibility of the upstream participants (i.e., doctors and hospitals) in the downstream in-home therapy sessions to monitory quality of care. Related-art computer systems and methods do not have the capability to provide that visibility.

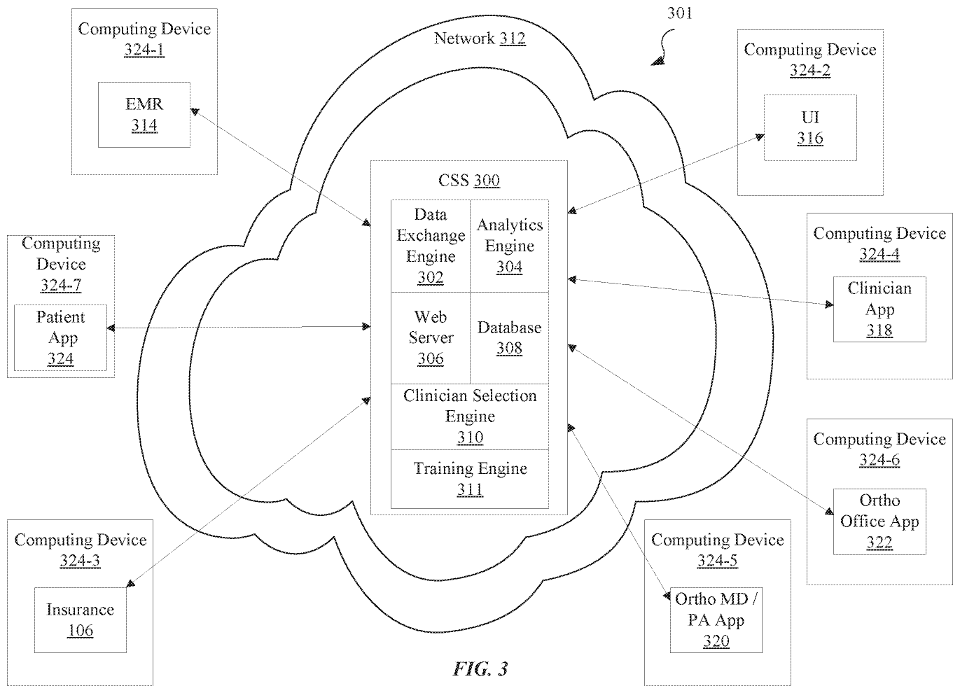

FIG. 3 shows, in block diagram form, a system 301 for coordination and control of in-home health care services, in accordance with at least some embodiments. In particular, FIG. 3 conceptually shows a central organization and structure that provides many services related to in-home therapy, the central services system 300 (in the drawing “CSS”). The central services system 300 may be a single computer system, a group of computer systems, cloud-based computer services, or combinations. In some embodiments, the central services system 300 may provide a multi-tenancy software as a service (SaaS) platform. The tenants (e.g., home health agencies) may be a group of users with access to the same shared privileges, data, and/or software functionality. For example, one home health agency may have numerous locations in the same state and/or different states in the country, and the various locations may have respective accounts that are tenants of the central services system 300 to leverage the software functionality provided therein. Providing the software functionality of the central services system 300 via a SaaS platform may enable customers to access the software functionality online via a subscription by self-provisioning.

The central services system 300 executes a plurality of related and communicatively interconnected software programs to provide the functionality. For example, the example central services system 300 may implement a data exchange engine 302, an analytics engine 304, a web server 306, a database 308, a clinician selection engine 310, and a training engine 311. Each example component of the central services system 300 will be discussed after introduction of the remaining portions of the example system 301.

The central services system 300 may communicatively couple to a host of external devices and systems, such as by way of the network 312. Network 312 may be a public network (e.g., connected to the Internet via wired (Ethernet) or wireless (WiFi)), a private network (e.g., a local area network (LAN), wide area network (WAN), virtual private network (VPN)), or a combination thereof. Numerous external devices 324 are depicted, and the computing devices 324 may be servers, desktop computers, laptop computers, smartphones, tablets, or any suitable computing devices including at least a processor, a memory, and a network interface device.

The central services system 300 may communicate with a host of EMR platforms 314, with EMR platform 314 representative of any of the previously discussed EMR platforms (e.g., EMR platforms 110 or 116 of FIG. 1). The EMR platforms may be implemented as computer instructions stored in a memory and executable by a processor of a computing device 324-1. In some embodiments, the computing device 324-1 may be associated with and operated by a Home Health Agency. As will be discussed more below, the central services system 300 in accordance with example embodiments is EMR platform agnostic, having the ability to electronically communicate with most if not all EMR platforms regardless of their underlying technology and/or association (e.g., EMR platform of an HHA, or an EMR platform of a Staffing Company).

The example central services system 300 may have a user interface 316 (in the drawing “UI”) through which the central services system 300 may be administered (e.g., credentialed access and administration by way of a series of web pages). While the user interface 316 is shown communicatively coupled to the central services system 300 by way of the network 312, the user interface 316 may likewise be directly coupled and/or within a local area network of some or all of the central services system 300. The user interface 316 may be implemented in computer instructions stored on a memory and executed by a processor of a computing device 324-2.

By way of the user interface 316, one or more administrators may perform day-to-day administration, such as adding and removing Clinicians, onboarding and administering HHAs into the system (e.g., including identification of the HHA EMR platform and credentials therefor), and onboarding and administering Staffing Companies into the system. The example central services system 300 communicatively couples to a computing device 324-3 of Insurance Providers 105/106, such as for billing services. The user interface 316 may be used to select Clinicians to provide in-home therapy for patients. Further, the user interface 316 may be used to transmit messages to other computing devices associated with entities (e.g., a Clinician) in the healthcare industry.

Still referring to FIG. 3, the example system further comprises a clinician application 318. Each Clinician (e.g., 1099 Clinician 114 or W2 Clinician 109 (FIG. 1)) operating with the example system will run an instance of the clinician application 318 on a computing device 324-4 (e.g., smartphone or tablet device). In some cases the clinician application 318 is indeed a standalone computer program communicatively coupled to the central services system 300, but in other cases the clinician application 318 could be a web-based access system. Such a web-based access system may have limited functionality compared to a computer program running on the portable computer system (e.g., web-based access may lack the ability to receive push notifications from the central services system 300).

Further still, the example system has an example orthopedic application 320 (shown in FIG. 3 as the “Ortho MD/PA App”). As mentioned above, the explanation of the example system is in reference to an orthopedic surgery, hence the reference to an orthopedic application 320; however, such an application shall be generic to any application to be used by a doctor, physician's assistant, or nurse practitioner concerned with or monitoring and controlling patient outcomes. As with the clinician application 318, the orthopedic application 320 may be run on a computer device 324-5 (e.g., smartphone or tablet device). In some cases the orthopedic application 320 is indeed a standalone computer program communicatively coupled to the central services system 300, but in other cases the orthopedic application 320 could be a web-based access system. Such a web-based access system may have limited functionality compared to a computer program running on the portable computer system (e.g., web-based access may lack the ability to receive push notifications from the central services system 300). Finally, the example system may comprise an orthopedic office application 322, such that the Doctor's Practice 103 (e.g., office administrator, nursing staff, etc.) may interact with the central services system 300 to assist on behalf of the doctor. The orthopedic office application 322 may be run on a computing device 324-6.

The example system has a patient application 324 that runs on a computing device 324-7 (e.g., smartphone or tablet device). In some cases the patient application 324 is indeed a standalone computer program communicatively coupled to the central services system 300, but in other cases the patient application 324 could be a web-based access system. Such a web-based access system may have limited functionality compared to a computer program running on the portable computer system (e.g., web-based access may lack the ability to receive push notifications from the central services system 300). The patient application 324 may be implemented in computer instructions and executed by a processing device of the computing device 324-7. The patient may use the patient application 324 to view their health information, therapy protocol, and/or communicate directly with a Physician, a Clinician, other patients, an insurance provider, or any other suitable entity in the healthcare industry.

Referring again to the central services system 300. The data exchange engine 302 is designed and constructed to exchange data with EMR platforms 314 and the Database 308. For example, the data exchange engine 302 may log into and retrieve new patient data from an HHA or Staffing Company via EMR 314 and place it within Database 308, where the patient data may then be accessed by user interface 316 and Clinician App 318, for example, and thus obviating the need for manual data entry for new patient onboarding. Likewise, the data exchange engine 302 may provide patient therapy data from Clinician App 318 to Database 308, to the EMR platform 314 for each in-home therapy session. The data exchange implemented by the data exchange engine 302 may take one of many suitable forms. In some cases a specifically designed application programming interface (API) may be invoked by the central services system 300. The example API may map the patient therapy data (e.g., stored in the database 308) in an organizational structure of the central services system 300 into a modified patient therapy data in an organizational structure required by the target EMR platform 314, and then communicate the modified patient therapy data to the first EMR platform using a first communication scheme of the first EMR platform. In yet still other cases, the data exchange engine 302 may include an artificial intelligence engine that has been trained and has the ability to parse the data fields of any EMR platform, and map the patient therapy data held in the database 308 into any structure used by an EMR platform 314.

Further, the data exchange engine 302 may provide a communication mechanism to enable direct real-time or near real-time messaging between entities in the healthcare industry. Communication mappings between the entities may be dynamically created to enable communication between the entities. For example, when patient therapy data for a patient is downloaded by the central services system 300, the data exchange engine 302 may identify a correlation between the identity of the patient and an identity of a Doctor that performed surgery on the patient. After a Clinician is assigned to provide the in-home therapy, the data exchange engine 302 may identify a correlation between the identity of the patient and an identity of the Clinician assigned to provide the in-home therapy. Based on the Clinician and the Doctor being correlated with the patient, the data exchange engine 302 may create a communication mapping between the identity of the Doctor and the identity of the Clinician. Further, the data exchange engine 302 may provide real-time or near real-time messaging between a computing device 324-5 of the Doctor and a computing device 324-4 of the Clinician. Other communication mappings may be created to enable direct real-time or near real-time communication between other entities (e.g., between a Clinician and a patient, between a patient and a Doctor, between a Clinician and a Scheduler, etc.), thereby creating an improved communication network in the healthcare industry.

The example web server 306 of the central services system 300 may provide web pages to remote devices to implement some or all of the underlying functionality. For example, the web server 306 may provide web pages to enable the user interface 316. Likewise, for clinicians, doctors, and doctor's offices not utilizing a standalone application, the web server 306 may provide various web pages to enable data exchange and other functionality with the central services system 300.

The database 308 is a database of information spanning all parts of the services provided by the central services system 300. For example, database 300 may store information about HHAs and Staffing Companies utilizing the services provided by the central services system 300. For each HHA and/or Staffing Company, the database 308 may store information regarding the EMR platform used by each HHA and/or Staffing Company (e.g., EMR platform identity, login credentials, and the like). The database 308 may store information about Clinicians working through the central services system 300. For example, the database 308 may contain information such as: Clinician name; Clinician contact information; Clinician licensing information (e.g., credential type, expiration date of credential); Clinician daily starting point; Clinician gender; Clinician ethnicity; Clinician outcomes for different surgery and therapy types; software communication addresses and handles; Clinician preference for certain Assistants; Clinician billing rate per visit; and the like. Likewise, the database 308 may store information regarding the Doctor and the Doctor's Practice and staff.

The example database 308 may store information regarding each patient, such as: patient name; patient contact information; patient's doctor identifier; patient address; patient therapy type; patient gender; patient ethnicity; patient's special request for a Clinician to be staffed; patient's special request to exclude a Clinician from being staffed; and the like. Moreover, as the in-home therapy sessions are provided, the database 308 may store the patient therapy data for each session, including as needed pictures of portions of the patient's body (e.g., incisions).

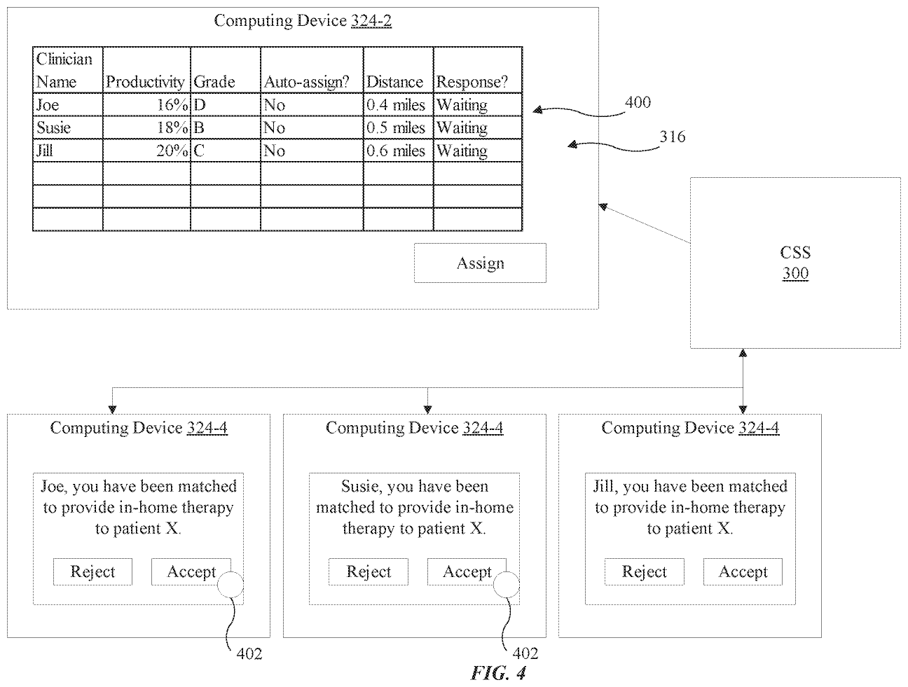

The example central services system 300 further comprises a Clinician selection engine 310. As the name implies, the Clinician selection engine 310 may, upon the overall central services system 300 receiving a new patient 100 (FIG. 1), assign a Clinician to the patient. In some example cases, the Clinician selection engine 310 may filter a number of criteria to arrive at the ideal Clinician assignment for a patient. Additional details pertaining to the Clinician selection engine 310 are described below with reference to FIGS. 4-8.

The example training engine 311 may create one or more machine learning models that are used by an artificial intelligence engine. The training engine 311 may train the machine learning models using training data that includes training inputs and corresponding target outputs. The training engine 311 may find patterns in the training data that map the training input to the target output (the answer to be predicted), and provide the machine learning models that capture these patterns. The machine learning models may comprise, e.g., a single level of linear or non-linear operations (e.g., a support vector machine [SVM]) or a deep network, i.e., a machine learning model comprising multiple levels of non-linear operations. Examples of such deep networks are neural networks including, without limitation, convolutional neural networks, recurrent neural networks with one or more hidden layers, and fully connected neural networks.

The specification now turns to an example workflow using the example system of FIG. 3. Consider a new patient who has just completed surgery and has been referred to an HHA for in-home therapy. The HHA has previously used the central services system 300, and thus already exists as a client, and the HHA again desires to utilize the central services system 300. Note that if the HHA indirectly uses the central services system 300 by way of a Staffing Company, EMR platform with which the central services system 300 interacts may be different (or the credentials may be different), but otherwise the improved workflow is largely the same. The central services system 300 is notified of the new patient (e.g., electronic mail message, facsimile or data exchange engine 302). Triggered by the initial contact, the central services systems 300, and in particular the data exchange engine 302, communicatively couples to the EMR platform 314 of the requesting entity and downloads the patient data which is then stored in the database 308 (possibly after mapping and manipulation into data organizational structures of the central services system 300). Since the data is electronically downloaded and stored, the possibility of transcription error is reduced or eliminated.

The central services system 300 then invokes the clinician selection engine 310 as discussed above. The central services system 300 may then provide relevant information to the selected Clinician by way of the Clinician application 318. The Clinician may then contact the patient to schedule the initial in-home therapy session. The in-home therapy session may include the Clinician, by way of the computing device 324-4 that runs the Clinician application 318, taking measurements of the patient's range of motion. The Clinician application 318 may then upload the range of motion data to the central services system 300 to be stored in database 308. Likewise, at the end of the initial in-home therapy session, the Clinician may obtain a signature of the patient again by way of the clinician application 318 running on the Clinician's computing device 324-4. The signature evidencing completion of a therapy session may then be uploaded to the central services system 300 and then database 308 as a prerequisite to billing. For the initial in-home therapy session, the Clinician may also schedule with the patient the follow-on in-home therapy sessions, in many cases the first follow-on in-home therapy session scheduled for the very next day. The proposed schedule is provided to the Clinician application 318, and the proposed therapy session schedule is uploaded to the central services system 300. During the in-home therapy session, the Clinician creates patient therapy data by inputting the relevant information (such as an individual joint's range of motion) directly into the Clinician application 318 running on the Clinician's computing device 324-4. The Clinician application 318 then transfers the patient therapy data to the central services system 300 for storage on the database 308. In the specific case of a Clinician providing therapy to orthopedic patients, either the Clinician application 318, or a second application available to the Clinician, may give the ability to perform orthopedic-specific task, such as taking pictures of the patient's incision for sending to the doctor.

A few notes before proceeding. First, in the example system the Clinician needs to interact with a single program: to provide one or more credentials (e.g., healthcare licenses), to agree to provide therapy to a patient; to get relevant information about the patient; to get a signature from the patient evidencing an in-home therapy session has taken place; in the case of orthopedic procedures possibly to provide visual information regarding patient's condition; to seek approval for follow-up sessions; and to create and provide patient therapy data. If all of the Clinician's contracting agencies (either HHAs or Staffing Companies) utilize the services of the central services system 300, then the Clinician has only one application and one set of login credentials with which to be concerned on a daily basis (rather than multiple EMR thin clients and associated login credentials).

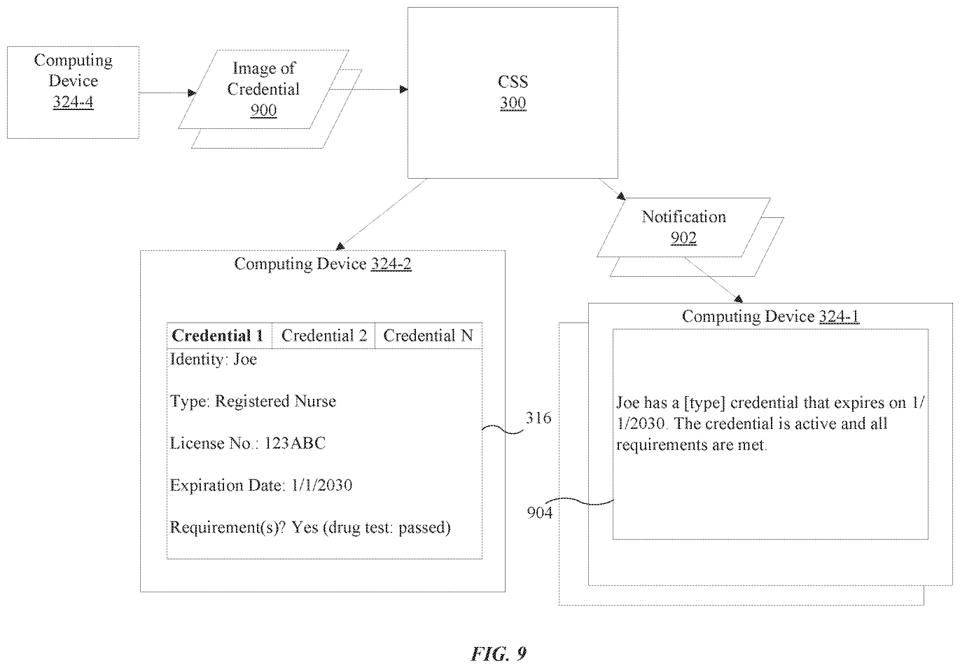

Further, the single application provided by the central services system 300 may function as a passbook that maintains and manages all the healthcare credentials issued to the Clinicians. Instead of having to provide their credentials to each home health agency, for example, the Clinician requests to join, the central services system 300 may transmit the stored credentials of the Clinician to the selected home health agencies. Further, when the credentials of the Clinicians expire or will expire in a certain time period, the central services system 300 may transmit a notification to the appropriate Clinician indicating such to enable the Clinician to update the credential at issue. The management of the credentials issued to Clinicians is further described below with reference to FIGS. 9-14.

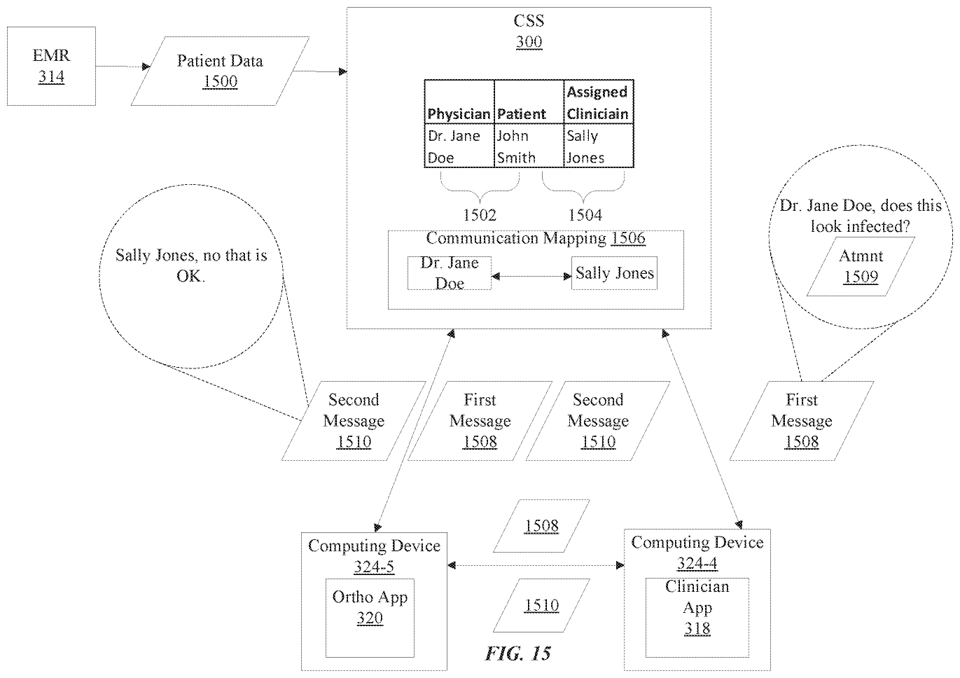

Triggered by the receipt of information from the clinician application 318, the central services system 300 may take several additional actions. With respect to the patient's therapy notes, or data, the central services system 300 may send the information to the orthopedic office application 322 and the orthopedic application 320 in real-time or near real-time from receipt. Thus, depending on the responsiveness of the doctor or the doctor's office, any feedback or instructions from the doctor can be communicated, possibly while the Clinician is still providing the in-home therapy session. Even if the responsiveness of the doctor is not fast enough to overlap with the in-home therapy session, the feedback and input may take place before the next therapy session or before the next in-home therapy has completed. Such is a significant improvement over related-art systems where the doctor may not even be provided the patient therapy data until after the in-home therapy has completed. The real-time or near real-time communication between computing devices of various entities (e.g., doctor, clinician, patient, scheduler, etc.) is further described below with reference to FIGS. 15-21.

The central services system 300 may also take action based on the proposed follow-on in-home therapy schedule. That is, upon receiving a proposed follow-on in-home therapy schedule, the central services system 300, and particularly the data exchange engine 302, may communicate with the EMR platform 314 to transfer the patient's data and then to seek approval for the follow-on session timing and number of sessions. Thus, it may be possible that the follow-on sessions are approved before the Clinician leaves the patient location. Even if the approval is not prior to departure of the Clinician (as caused by delays on approver's EMR platform) the chance of approval arriving in good time before the proposed follow-on session is greatly improved, thus reducing or eliminating the chances of missed next-day sessions.

In situations where the contract agency (e.g., HHA or Staffing Company) requires the patient therapy data for the first in-home therapy session before approving follow-on sessions, the example central services system 300 may, as soon as the patient therapy data arrives from the Clinician application 318, subject the patient therapy data to a programmatic quality assurance check, and then provide the data with the EMR Platform 314. In some example embodiments the patient therapy data may be uploaded to the EMR platform 314 within seconds of the patient therapy data being uploaded by the Clinician application 318, along with the information regarding the follow-on session timing and number of sessions. Even if the approval for the follow-on sessions is after departure of the Clinician (as caused in part by timing of the Clinician inputting the patient therapy data and/or the approver's EMR platform) the chance of approval arriving in good time before the proposed follow-on session is greatly improved, thus reducing or eliminating the chances of missed next day sessions.

The process of the Clinician providing in-home therapy sessions thus repeats for the number of sessions, each time the Clinician possibly records patient's progress by way of the clinician application 318, getting a signature by way of the clinician application 318, and creating and uploading patient therapy data by way of the clinician application 318. For each session, the orthopedic application 320 and/or orthopedic office application 322 are provided the patient therapy data, enabling near real-time feedback and guidance to the clinician. Moreover, the automated systems reduce transmission time and organizational issues to mere seconds of computer time, greatly reducing the billing and collection cycle.

As mentioned above, various insurance companies and agencies are moving toward a pay-for-performance model of reimbursement for services. However, the related-art largely manual- and paper-based implementation does not provide sufficient data with which to determine quality of patient outcomes. Likewise, related-art systems do not have the ability to identify differences in workflows that may result in better overall patient outcomes if such differences were identified and disseminated. That is, the existing computer systems do not have the ability to provide sufficient metrics for pay-for-performance systems. However, the example system of FIG. 3, in addition to improving operation of related-art computer systems, also has the ability to improve another technological field, namely data analytics to identify and rate outcomes, and identify and disseminate workflow improvements to improve outcomes for all patients.

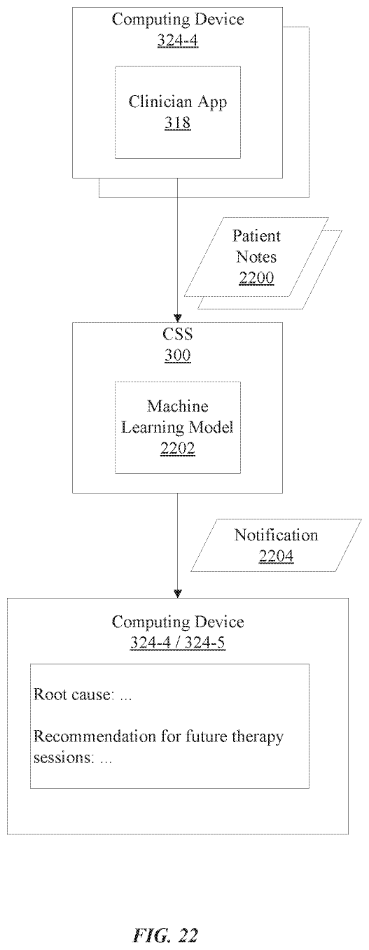

In particular, consider that the example system of FIG. 3 receives patient referrals from 100 doctors performing 30 surgeries a month, and that each surgery results in 10 in-home therapy sessions. With just this relatively small number of doctors, the example system of FIG. 3 may obtain as many as 30,000 sets of patient therapy data (hereafter just “patient notes”) a month regarding in-home therapy, and thus some 360,000 patient notes a year. In accordance with at least some embodiments, the example central services system 300 may perform patient, vendor, and/or doctor analysis (e.g., the analytics engine 304) on the data (e.g., stored in database 308). Such data analytics is further described below with reference to FIGS. 22-26. The specification now turns to a discussion of Clinician selection.

### Clinician Selection

The example high-level workflow discussed above assumed selection of an appropriate Clinician so as not to unduly complicate the discussion. Now, however, the specification turns to example embodiments of selecting a Clinician. Initially, the Clinician selection engine 310 may generate a group of candidate Clinicians from a data set of Clinicians (e.g., stored in the database 308). The selection may be based on one or a host of criteria, such as: the location of a Home Health Agency; the location of the Clinicians associated with the Home Health Agency; a status of the Clinicians (e.g., active, inactive); a special request to assign a certain Clinician or to exclude a certain Clinician from being assigned; an EMR platform on which the Clinician is trained; the discipline of the type of therapy assigned for the patient; the distance to the patient from each Clinician's daily starting point; gender of patient and/or Clinician; ethnicity of patient and/or Clinician; religious affiliation of patient and/or Clinician; statistical outcomes of therapy type for the Clinician; mean distance to other patients of the Clinician; utilization of each Clinician relative to their overall capacity to work for a certain time period (e.g., a week); a billing rate the Clinician charges per visit; a rating of the Clinician; whether the referral indicates an assistant for the Clinician is allowed; a spoken language of the patient and/or Clinician; a specialty of the Clinician; type of employment for the Clinician (e.g., full-time, part-time, 1099, etc.); factors related to other patients simultaneously needing in-home therapy; and factors related to other Clinician. Once the group of candidate Clinicians is generated, the group may be ranked and sorted in a particular order. The sorting may be based on one or more of the above-listed criteria. For example, candidate Clinicians that have the lowest productivity and/or are closest to the patient may be ranked higher in the result set than other candidate Clinicians.

The Clinician selection engine 310 may contact each member of the group of candidate Clinicians (e.g., text, electronic mail message, push notification to the Clinician application 318). In some embodiments, the clinician selection engine 310 may contact each Clinician according to rank until a Clinician agrees to accept the patient. Each candidate Clinician contacted may express interest or not, and thus the Clinician selection engine 310 may thus receive indications of interest from a subset of the group of candidate Clinicians. From the subset of the group of candidate Clinicians, the Clinician selection engine 310 may assign the patient to the Clinician for in-home therapy. In other cases the Clinician may be selected by way of a manager from the user interface 316.

The disclosed embodiments may improve a user's experience using a computing device by providing candidate Clinicians that are specifically matched for a particular patient without the user having to search through numerous Clinician profiles. To that end, the result set of the matched candidate Clinicians to a patient may enable a user (e.g., Scheduler) to identify and staff a Clinician from an initial list displayed on a screen of the user interface 316 without having to search through multiple screens. The candidate Clinicians that are best suited to provide in-home therapy for a specific patient may be displayed using the disclosed techniques and the Scheduler may not request additional searches be performed by the central services system 300. As a result, the disclosed techniques may reduce network bandwidth consumption, memory consumption, and/or processing consumption.

To illustrate, FIG. 4 shows, in block diagram form, an example workflow for generating a group of candidate Clinicians and transmitting notifications to the group of candidate Clinicians, in accordance with at least some embodiments. When a referral is received from a healthcare facilitator, such as a Doctor's office, hospital, or the like, the central services system 300 may electronically download the information provided in the referral from an EMR platform 314. The information specified in the referral may include an identity of the patient, an identity of the doctor that provided healthcare to the patient, a discipline of a type of therapy to be provided by a Clinician, a zip code of the patient, when the in-home therapy should start, special requests by the patient (e.g., for a specific gender, spoken language, Clinician, etc.), and the like. The Clinician selection engine 310 executed by the central services system 300 may generate a group of candidate Clinicians 400 that are matches for the patient based on the information in the referral and one or more of the criteria described above.

As depicted, the group of candidate Clinicians 400 may be generated by the central services system 300 and transmitted to the computing device 324-2 of a Scheduler. The group of candidate Clinicians 400 may be presented in the user interface 316 on the computing device 324-2. The group of candidate Clinicians 400 includes Clinicians “Joe,” “Susie,” and “Jill.” The group is ranked based on one or more factors. For example, Joe has the lowest productivity (e.g., 16%), which may refer to his utilization rate for a time period compared to his overall capacity to work that time period, a low grade (e.g., rating), and is the closest relative to the location of the patient (e.g., 0.4 miles away). In some embodiments, the ranking may be based on additional criteria, including: the employment type of the Clinician, whether the Clinician will work with an assistant, a billing rate per visit, a date the Clinician prefers to visit the patient, etc. As depicted, each Clinician has chosen to not be automatically assigned when they are matched to a patient referral. Also, a column may be included for a type of response received from the Clinicians. The response may be “waiting” when no response is received yet, “accepted” when the Clinician accepts the in-home therapy, “rejected” when the Clinician rejects the in-home therapy, and/or “assigned” when the Scheduler or auto-assignment assigns the Clinician to provide the in-home therapy.

Accordingly, when they are matched, notifications are transmitted to the Clinician applications 318 executing on their computing devices 324-4. The notifications may be presented on the computing devices 324-4, as depicted, and may state that the particular Clinician has been matched to provide in-home therapy to the patient (e.g., patient X). Further, the notification may provide one or more graphical user interface elements to allow the user to accept or reject the in-home therapy for the patient. As depicted by circles 402, both Joe and Susie selected the graphical user interface element to accept the in-home therapy for the patient.

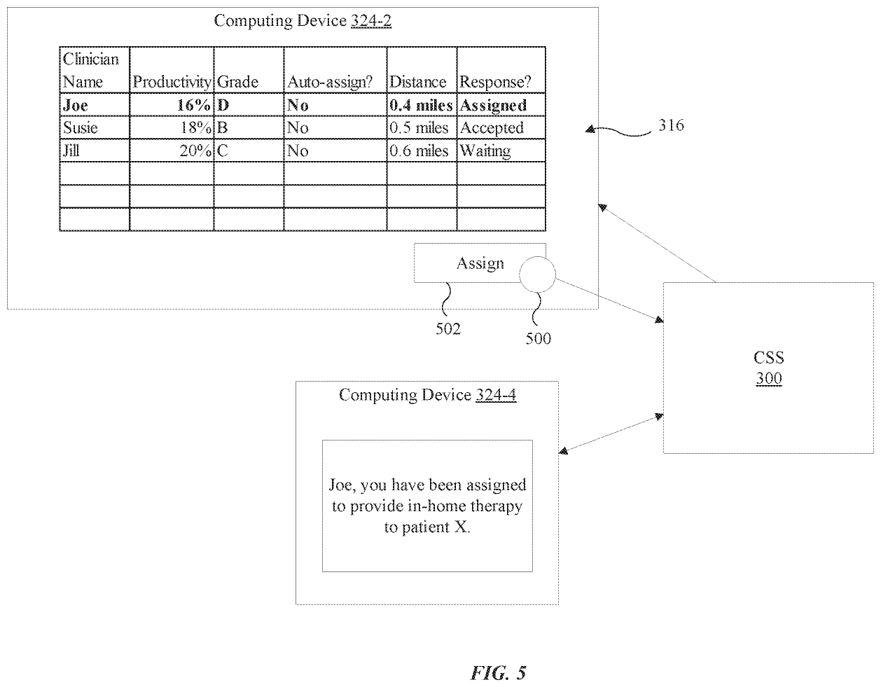

FIG. 5 shows, in block diagram form, an example workflow for a Scheduler selecting a candidate Clinician to assign for in-home therapy for a patient, in accordance with at least some embodiments. As depicted, Joe and Susie accepted the assignment to provide the in-home therapy for the patient. For example, the “Response?” column in the user interface 316 of the computing device 324-2 of the Scheduler displays “Accepted” for Susie. Also, since Jill has not yet responded to the notification, the “Response?” column for her indicates that the central services system 300 is still “Waiting” for her response. Since neither Joe nor Susie chose to be automatically assigned when matched to a patient referral, the Scheduler may choose the Clinician to assign to the patient referral. The Scheduler selected (as shown by circle 500) Joe to provide the in-home therapy for the patient by selecting his name and selecting the “Assign” button 502. The “Response?” column for Joe may indicate that he has been “Assigned” to the in-home therapy for the patient. Selecting the button 502 may cause the computing device 324-2 to transmit the selection to the central services system 300. The central services system 300 may transmit a notification to Joe's computing device 324-4 and the notification may be presented on a display of Joe's computing device 324-4. The notification may indicate that Joe has been assigned to provide the in-home therapy to patient X.

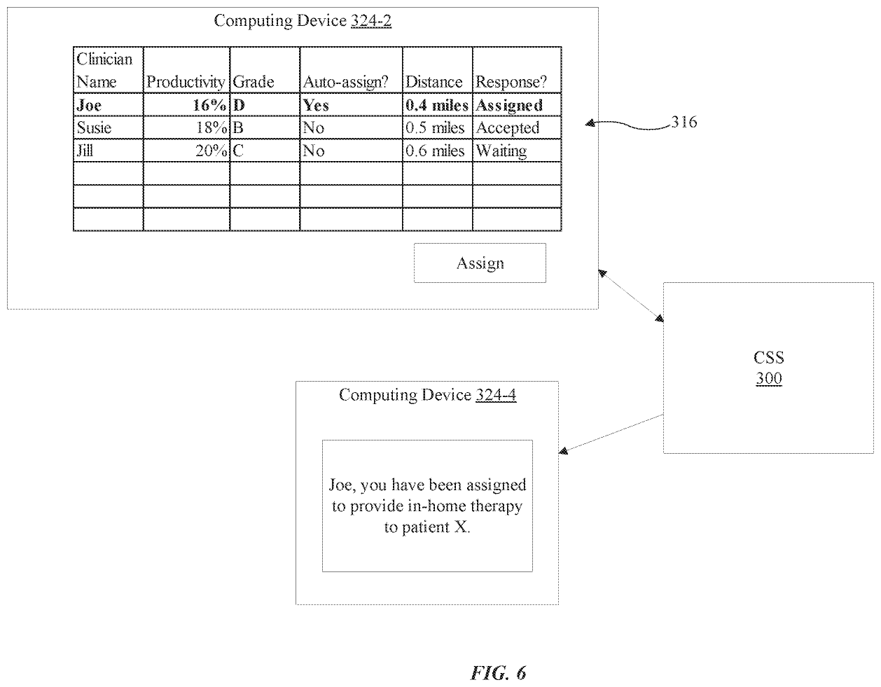

FIG. 6 shows, in block diagram form, an example workflow for automatic assignment of a Clinician to provide in-home therapy for a patient, in accordance with at least some embodiments. As depicted, the group of candidate Clinicians is generated by the central services system 300 and transmitted to the computing device 324-2 of the scheduler for presentation in the user interface 316. The Clinician, Joe, selected to be automatically assigned to a patient referral whenever he is matched to a patient referral. For example, the “Auto-assign?” column for Joe indicates “Yes”. Instead of the Scheduler selecting who to assign to the patient referral, Joe is automatically assigned to the patient referral when he is matched. Thus, the “Response?” column indicates that Joe is “Assigned” to the patient referral. The central services system 300 may transmit a notification to Joe's computing device 324-4 that indicates that he has been assigned to provide the in-home therapy to the patient.

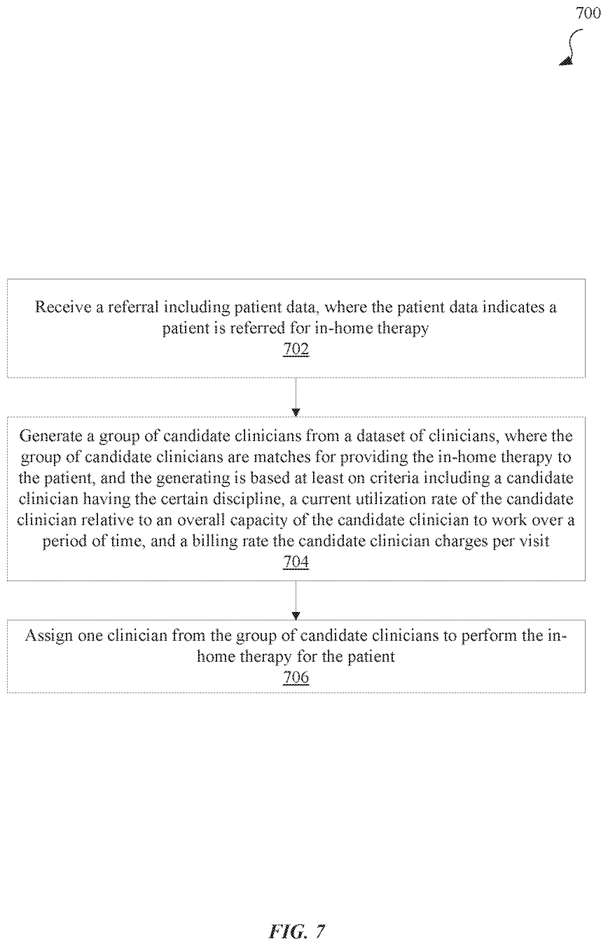

FIG. 7 shows an example method 700 for generating a group of candidate Clinicians and assigning a Clinician to perform in-home therapy for a patient, in accordance with at least some embodiments. The method 700 may be performed by processing logic that may include hardware (circuitry, dedicated logic, etc.), firmware, software, or a combination of them. The method 700 and/or each of their individual functions, subroutines, or operations may be performed by one or more processing devices. For example, the method 700 may be implemented as computer instructions executable by a processing device of the central services system 300. In certain implementations, the method 700 may be performed by a single processing thread. Alternatively, the method 700 may be performed by two or more processing threads, each thread implementing one or more individual functions, routines, subroutines, or operations of the methods.

At operation 702, the processing device may receive a referral including patient data, where the patient data indicates a patient is referred for in-home therapy. The patient data may be obtained from an application programming interface of an EMR platform 314. For example, after a patient undergoes surgery at a hospital, the doctor that performed the surgery may submit a referral to a Home Health Agency for the patient to receive in-home therapy. The patient data in the referral may be stored in an EMR platform 314 of the hospital and/or the Home Health Agency, and the patient data in the referral may be received by the processing device. The patient data may include other information as well, such as an identity of the doctor that provided healthcare to the patient, whether the patient has special requests, a discipline of the in-home therapy to be provided, whether the patient requests the Clinician to have an assistant, a request for a specific Clinician to be assigned for the in-home therapy, a request for a specific Clinician to be excluded from being assigned for the in-home therapy, and the like.

At operation 704, the processing device may generate a group of candidate Clinicians from a dataset of Clinicians, where the group of candidate Clinicians are matches for providing in-home therapy to the patient. The generating of the group of candidate Clinicians may be based at least one criteria including a candidate Clinician having the certain type of discipline for the in-home therapy, location(s) of a Home Health Agency the candidate Clinician works with, a current utilization rate of the candidate Clinician relative to an overall capacity of the candidate Clinician to work over a period of time, a billing rate the candidate Clinician charges per visit, a distance to the patient from a daily starting point of the candidate Clinician, a gender of the patient and/or the candidate Clinician, an ethnicity of the patient and/or the candidate Clinician, a spoken language of the patient and/or the candidate Clinician, whether the candidate Clinician is trained on an EMR platform used by a source from which the referral was received, whether the candidate Clinician is trained in a specialty (e.g., patient is chronically ill, the patient has a neurological disorder, etc.) needed to treat the patient, whether an assistant is to aid the candidate Clinician in the in-home therapy, special requests of the patient for certain candidate Clinicians to provide the in-home therapy or to be excluded from providing the in-home therapy, and/or a status of the candidate Clinician. The location of the Home Health Agency may be specified because Home Health Agencies may have numerous locations in different states around the country and the one nearest to the patient and/or Clinician may be identified to find the appropriate Clinicians.

In some embodiments, the group of candidate Clinicians may be ranked in an order based on one or more of the criteria described above. Additional criteria may be considered when performing the ranking, such as a type of employment of the candidate Clinician (e.g., full-time, part-time, 1099, etc.). Also, the processing device may generate a rating for each candidate Clinician in the group of candidate Clinicians, and the rating may be considered when ranking the group of candidate Clinicians in the order. Further, one or more of the criteria used to generate and/or rank the group of candidate Clinicians may be weighted. For example, a weight may be added to the criteria related to the current utilization rate of the candidate Clinician relative to the overall capacity of the candidate Clinician to work over a period of time. The weight may increase a probability that the candidate Clinicians who are under-utilized relative to their overall capacity to work over the period of time are included in the group of candidate Clinicians.

In some embodiments, the generating of the group of candidate Clinicians may be performed by an artificial intelligence engine using one or more machine learning models. The machine learning models may be trained to receive patient data as input and generate the group of candidate Clinicians as output. The machine learning models may be supplied a set of patient data and access a database of Clinician data. The machine learning models may select a group of candidate Clinicians based on the patient data received in the referral and the database of Clinician data.

At operation 706, the processing device may assign one Clinician from the group of candidate Clinicians to perform the in-home therapy for the patient. In some embodiments, when the candidate Clinicians have indicated a preference to be automatically assigned to providing in-home therapy to a matched patient, the processing device may automatically assign the one Clinician from the group of candidate Clinicians. If the candidate Clinician has not indicated a preference for being automatically assigned, the Scheduler may select which one of the candidate Clinicians to assign to provide the in-home therapy to the patient. Further, in some embodiments, the artificial intelligence engine including the one or more machine learning models may select the one Clinician from the group of candidate Clinicians to be assigned to provide the in-home therapy for the patient.

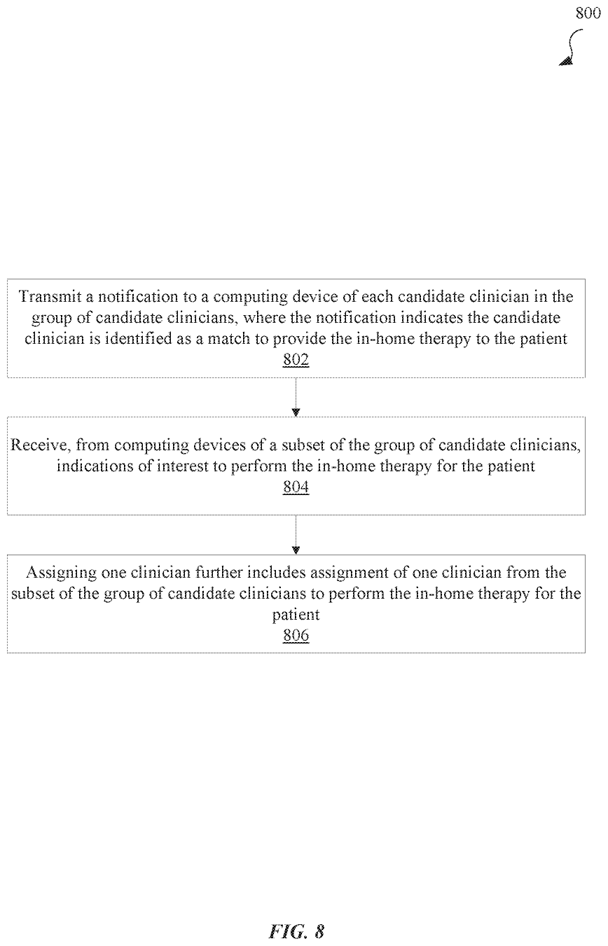

FIG. 8 shows an example method 800 for transmitting notifications to a group of candidate Clinicians, in accordance with at least some embodiments. Method 800 includes operations performed by processing devices of the central services system 300 of FIG. 3. In some embodiments, one or more operations of the method 800 are implemented in computer instructions executable by a processing device of the central services system 300. The method 800 may be performed in the same or a similar manner as described above in regard to method 700 of FIG. 7.

At operation 802, the processing device may transmit a notification to a computing device 324-4 of each candidate Clinician in the group of candidate Clinicians, where the notification indicates the candidate Clinician is identified as a match to provide the in-home therapy to the patient. The transmitting may include sending a text message to the computing device 324-4 of each candidate Clinician, sending an electronic mail message to the computing device 324-4 of each candidate Clinician, and/or sending a push notification to an application running on the computing device 324-4 of each candidate Clinician. The notification may be presented on the display of the candidate Clinicians' computing devices 324-4 executing the Clinician application 318. The candidate Clinicians may be enabled to accept or reject providing the in-home therapy for the patient via one or more graphical user interface elements presented on the computing devices 324-4.

At operation 804, the processing device may receive, from computing devices 324-4 of a subset of the group of candidate Clinicians, indications of interest to perform the in-home therapy for the patient. At operation 806, the processing device may assign one Clinician from the subset of the group of candidate Clinicians to perform the in-home therapy for the patient. In some embodiments, if the one Clinician indicated a preference for automatic assignment, the central services system 300 may automatically assign the one Clinician to provide the in-home therapy for the patient. If the one Clinician did not provide an indication of a preference for automatic assignment, the Scheduler may choose the one Clinician for assignment after the one Clinician provides the indication of interest from the computing device 324-4.

### Electronic Credential Management

The central services system 300 may operate as a single source to maintain and manage the credentials issued to Clinicians by various agencies. The credentials may pertain to healthcare, such as nursing licenses, medical licenses, physical therapy licenses, and the like. The management by the central services system 300 may be electronic and may not involve administration by a user. The Clinicians may provide an image of their credential(s) to the central services system 300 using the Clinician application 318 executing on their computing device 324-4. The central services system 300 may extract certain information from the image of the credential, such as an expiration date of the credential, an identity of the Clinician issued the credential, a type of the credential, an identifier (e.g., license number) of the credential, and so forth. In some embodiments, the central services system 300 may validate the credential by communicating with a computing device of an agency that issued the credential. For example, the central services system 300 may make a call to an application programming interface of the issuing agency to validate the credential provided by the Clinician.

In some embodiments, the central services system 300 may provide notifications to various Home Health Agencies with which the Clinician is associated. The Clinician may just upload the image of their credential to the central services system 300 instead of having to transmit the image to the numerous Home Health Agencies with which they are associated. Accordingly, the computing device 324-4 may consume less network bandwidth by communicating with a single source (e.g., the central services system 300) instead of numerous computing devices of the Home Health Agencies. Further, the central services system 300 may electronically maintain and manage the credentials for Clinician, thereby eliminating potential human error when entering information.

In some embodiments, when the expiration date of a credential is going to expire within a certain time period (e.g., days, weeks, months), the central services system 300 may transmit a notification to a computing device 324-4 of the Clinician associated with the credential. The notification may instruct the Clinician to provide an updated credential before the one on file expires. If the credential expires, the Clinician may be prevented from being staffed to a patient referral. That is, the Clinician may be excluded from the group of candidate Clinicians when the central services system 300 is selecting and assigning a Clinician to provide in-home therapy for a patient. If the Clinician provides an updated credential, the central services system 300 may communicate the same to the Home Health Agencies with which the Clinician is associated. In some embodiments, the notification to the Home Health Agencies may just indicate that the Clinician has a valid new credential and may not transmit the actual image of the credential. Thus, in those embodiments, the memory footprint of the computing devices of the Home Health Agencies may be reduced by not storing the image of the credentials. Accordingly, the user experience of engaging with Home Health Agencies for a Clinician may be greatly improved using the disclosed central services system 300, as it provides electronic maintenance and management of all credentials of a Clinician. Essentially, the central services system 300 may be compared to a passbook of credentials for the Clinicians.

FIG. 9 shows, in block diagram form, an example workflow for electronic credential management, in accordance with at least some embodiments. A Clinician captures an image 900 of a credential using the computing device 324-4, for example. In some embodiments, the image 900 may be stored on another device and the image 900 may be downloaded by the computing device 324-4. The image of the credential 900 may be communicated to the central services system 300. The central services system 300 may perform one or more data extraction techniques by processing the image 900 of the credential. For example, in some embodiments, the central services system 300 may perform object character recognition (OCR) on the image 900 of the credential to extract an expiration date of the credential, an identity of the Clinician, a type of credential, an identifier of the credential, etc. In some embodiments, the central services system 300 may input the image into an artificial intelligence engine that uses one or more machine learning models to identify and extract the pertinent information discussed above. The extract information may be associated with the Clinician that provided the image 900 and stored in the database 308, for example.

Further, the central services system 300 may transmit a notification to a computing device 324-2 of a Scheduler and/or administrator to be presented on the user interface 316. The notification may include the information that was electronically extracted from the image 900 of the credential. The data associated with all of the credentials of the particular Clinician may be presented on the user interface 316. As depicted, the user interface 316 presents “Credential 1” as being selected, and the information for “Credential 1” is presented. For example, the information includes “Identity: Joe,” “Type: Registered Nurse,” “License No.: 123ABC,” “Expiration Date: Jan. 1, 2030,” and “Requirement(s)? Yes (drug test: passed).” The information for the other credentials “Credential 2” and “Credential 3” may also be viewed by the user of the computing device 324-2 if the appropriate tab is selected. Any suitable information associated with the credentials may be presented.

Upon receiving the image 900 and processing the image 900, the central services system 300 may transmit a notification 902 to the computing devices 324-1 associated with the Home Health Agencies with which the Clinician is associated. If the Clinician is associated with more than one Home Health Agency, the central services system 300 may transmit the notification to each of the computing devices 324-1 of the Home Health Agencies. The notification 902 may be presented on a user interface 904 of the computing device 324-1. In some embodiments, the notification includes a message that may state “Joe has a [type] credential that expires on Jan. 1, 2030. The credential is active and all requirements are met.” In some embodiments, the image 900 of the updated credential is not transmitted to the computing devices 324-1 of the Home Health Agencies. In some embodiments, the image 900 of the updated credential is transmitted to the computing devices 324-1 with the notification 902.

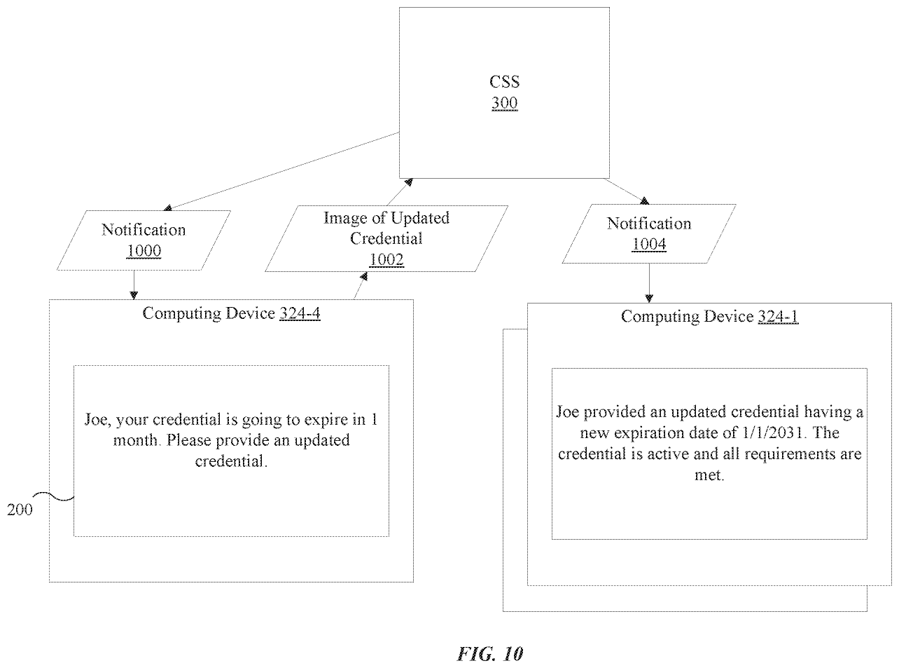

FIG. 10 shows, in block diagram form, an example workflow for providing a notification to a Clinician when a credential is going to expire within a time period, in accordance with at least some embodiments. As depicted, the central services system 300 may determine that a credential of a Clinician is going to expire within a certain period of time (e.g., days, weeks, months). The central services system 300 may transmit a notification 1000 to the computing device 324-4 that is executing the Clinician application 118. The notification 1000 may include an instruction that states “Joe, your credential is going to expire in 1 month. Please provide an updated credential.” The Clinician may use the Clinician application 118 to capture an image 1002 of an updated credential. The image 1002 of the updated credential may be transmitted to the central services system 300 from the computing device 324-4 executing the Clinician application 118. The central services system 300 may receive the 1002 and process the image 1002 to extract the expiration date, the identity of the Clinician, the type of credential, the identifier of the credential, and so forth. The central services system 300 may determine whether the credential is active based on whether the expiration date has expired and/or whether the issuing agency confirms the credential is valid.

The central services system 300 may transmit a notification 1004 to the computing devices 324-1 of the Home Health Agencies with which the Clinician is associated. The notification may indicate that “Joe provided an updated credential having a new expiration date of Jan. 1, 2031. The credential is active and all requirements are met.” In some embodiments, the image 1002 of the updated credential is not transmitted to the computing devices 324-1 of the Home Health Agencies. In some embodiments, the image 1002 of the updated credential is transmitted to the computing devices 324-1 with the notification 1004.

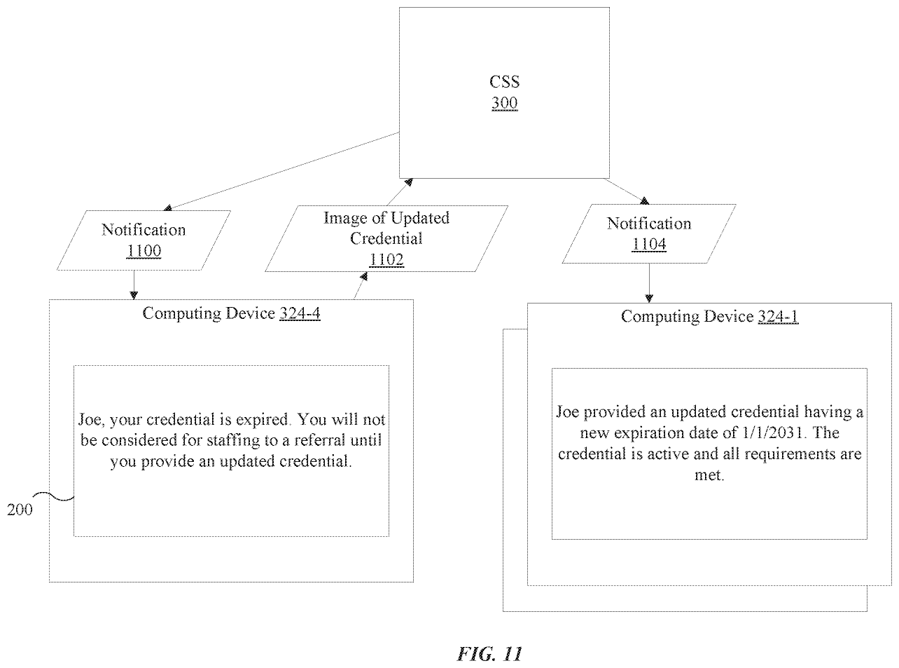

FIG. 11 shows, in block diagram form, an example workflow for providing a notification to a Clinician when a credential is expired, in accordance with at least some embodiments. As depicted, the central services system 300 may determine that a credential of a Clinician is expired. The central services system 300 may transmit a notification 1100 to the computing device 324-4 that is executing the Clinician application 118. The notification 1100 may include an instruction that states “Joe, your credential is expired. You will not be considered for staffing to a referral until you provide an updated credential.” The Clinician may use the Clinician application 118 to capture an image 1102 of an updated credential. The image 1102 of the updated credential may be transmitted to the central services system 300 from the computing device 324-4 executing the Clinician application 118. The central services system 300 may receive the 1102 and process the image 1102 to extract the expiration date, the identity of the Clinician, the type of credential, the identifier of the credential, and so forth. The central services system 300 may determine whether the credential is active based on whether the expiration date has expired and/or whether the issuing agency confirms the credential is valid.

The central services system 300 may transmit a notification 1104 to the computing devices 324-1 of the Home Health Agencies with which the Clinician is associated. The notification may indicate that “Joe provided an updated credential having a new expiration date of Jan. 1, 2031. The credential is active and all requirements are met.” In some embodiments, the image 1102 of the updated credential is not transmitted to the computing devices 324-1 of the Home Health Agencies. In some embodiments, the image 1102 of the updated credential is transmitted to the computing devices 324-7 with the notification 1104.

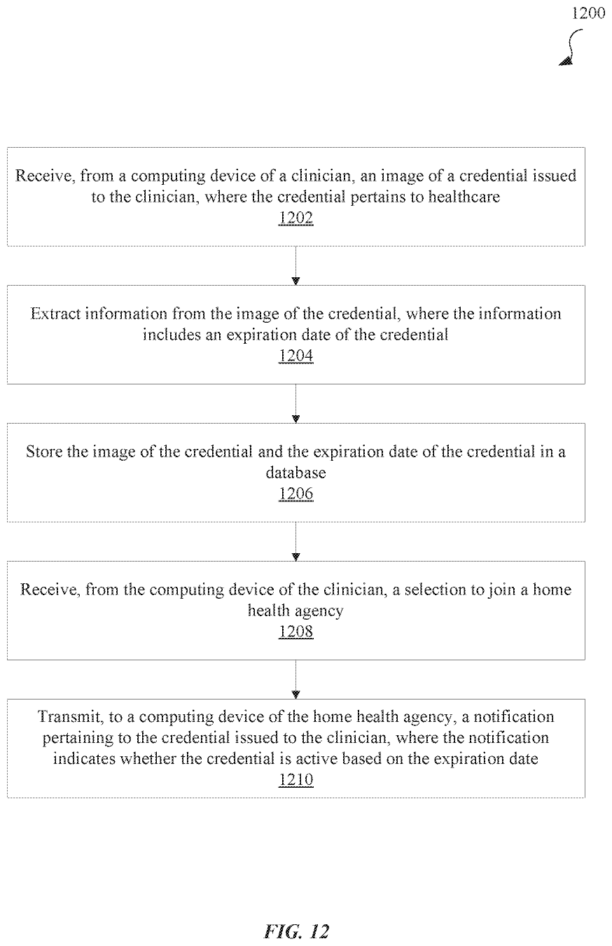

FIG. 12 shows an example method 1200 for electronic credential management, in accordance with at least some embodiments. Method 1200 includes operations performed by processing devices of the central services system 300 of FIG. 3. In some embodiments, one or more operations of the method 1200 are implemented in computer instructions executable by a processing device of the central services system 300. The method 1200 may be performed in the same or a similar manner as described above in regard to method 700 of FIG. 7.

At operation 1202, the processing device may receive, from a computing device 324-4 of a Clinician, an image of a credential issued to the Clinician, where the credential pertains to healthcare. The image may be captured using a camera of the computing device 324-4 or may be retrieved from storage on another computing device. The image may include pixels having various red, green, and blue (RGB) values.

At operation 1204, the processing device may extract information from the image of the credential, where the information includes an expiration date of the credential. Extracting the information may include performing OCR on the image of the credential to extract the expiration date of the credential. In some embodiments, extracting the information may include inputting the image of the credential into a machine learning model trained to extract the expiration date from the image of the credential. The machine learning model may have been trained using training data of images of numerous credentials that include a label at least identifying the expiration date on the image. Accordingly, the machine learning model may be capable of recognizing and extracting the expiration date of the provided image of the credential.

At operation 1206, the processing device may store the image of the credential and the expiration date of the credential in a database 308. In some embodiments, the processing device may validate the credential is issued to the Clinician by communicating with an application programming interface (API) of an issuing agency that issued the credential to the clinician. The API of the issuing agency may validate that the Clinician was or was not issued the credential and may validate the identifier of the credential, the expiration date of the credential, and/or any other suitable information pertaining to the credential. If the credential is not valid, the processing device may transmit a notification to the computing device 324-4 executing the Clinician application 318 that indicates the credential is not valid and instructs the Clinician to provide another credential.

At operation 1208, the processing device may receive, from the computing device 324-4 of the Clinician, a selection to join a Home Health Agency. Any Home Health Agency that is registered with the central services system 300 may be available to be joined by the Clinician via the Clinician application 318.

At operation 1210, the processing device may transmit, to a computing device 324-1 of the Home Health Agency, a notification pertaining to the credential issued to the clinician, where the notification indicates whether the credential is active based on the expiration date. In some embodiments, the notification may include information pertaining to the credential, such as the identifier of the credential, the identity of the Clinician, the expiration date of the credential, and the like. In some embodiments, the notification may include the image of the credential. In some embodiments, the processing device may determine, based on the expiration date, that the credential is expired. As a result, the processing device may prevent the Clinician from being considered for staffing to a patient referral. The processing device may transmit a notification to the computing devices 324-1 Home Health Agencies with which the Clinician is associated that indicates that the credential is expired. Further, the processing device may transmit a notification to the computing device 324-4 of the Clinician to instruct the Clinician to provide an updated credential.

In some embodiments, the central services system 300 may function as a passbook storing all credentials pertaining to healthcare for the Clinician. Accordingly, the processing device may receive, from the computing device 324-4 of the Clinician, a second image of a second credential issued to the Clinician, where the second credential pertains to healthcare. The processing device may extract second information from the second image of the second credential, where the second information includes an expiration date of the second credential. The processing device may store the second image of the second credential and the expiration date of the second credential in the database 308 for the Clinician. The processing device may transmit, to the home health agency, a second notification pertaining to the second credential provided by the clinician, wherein the second notification indicates whether the second credential is active based on the expiration date of the second credential

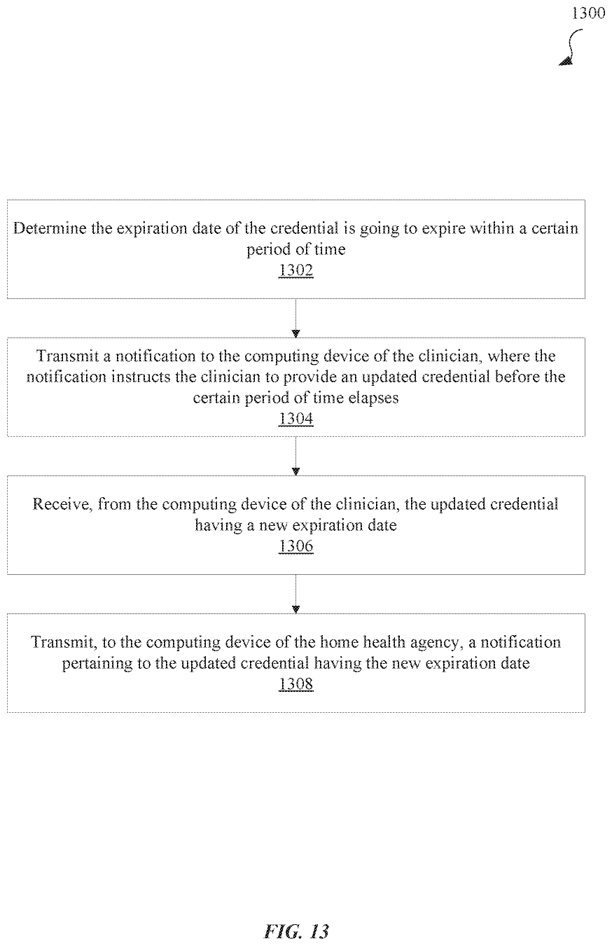

FIG. 13 shows an example method 1300 for providing a notification to a Clinician when a credential is going to expire within a time period, in accordance with at least some embodiments. Method 1300 includes operations performed by processing devices of the central services system 300 of FIG. 3. In some embodiments, one or more operations of the method 1300 are implemented in computer instructions executable by a processing device of the central services system 300. The method 1300 may be performed in the same or a similar manner as described above in regard to method 700 of FIG. 7.

At operation 1302, the processing device may determine the expiration date of the credential is going to expire within a certain time period. The time period may be any configurable amount, such as within days, weeks, months, etc.

At operation 1304, the processing device may transmit a notification to the computing device 324-4 of the Clinician, where the notification instructs the Clinician to provide an updated credential before the certain time period elapses. If the certain time period elapses, and the credential expires, the Clinician may be prevented from being considered for staffing to a patient referral until an updated credential is provided.

At operation 1306, the processing device may receive, from the computing device 324-4 of the Clinician, the updated credential having a new expiration date. In particular, the processing device may receive an image of the updated credential from the computing device 324-4 of the Clinician.

At operation 1308, the processing device may transmit, to the computing device 324-4 of the Home Health Agency, a notification pertaining to the updated credential having the new expiration date. The notification may include the image of the updated credential and/or various information pertaining to the updated credential, such as the type of the updated credential, an identity of the Clinician, an identifier of the updated credential, the expiration date, etc.

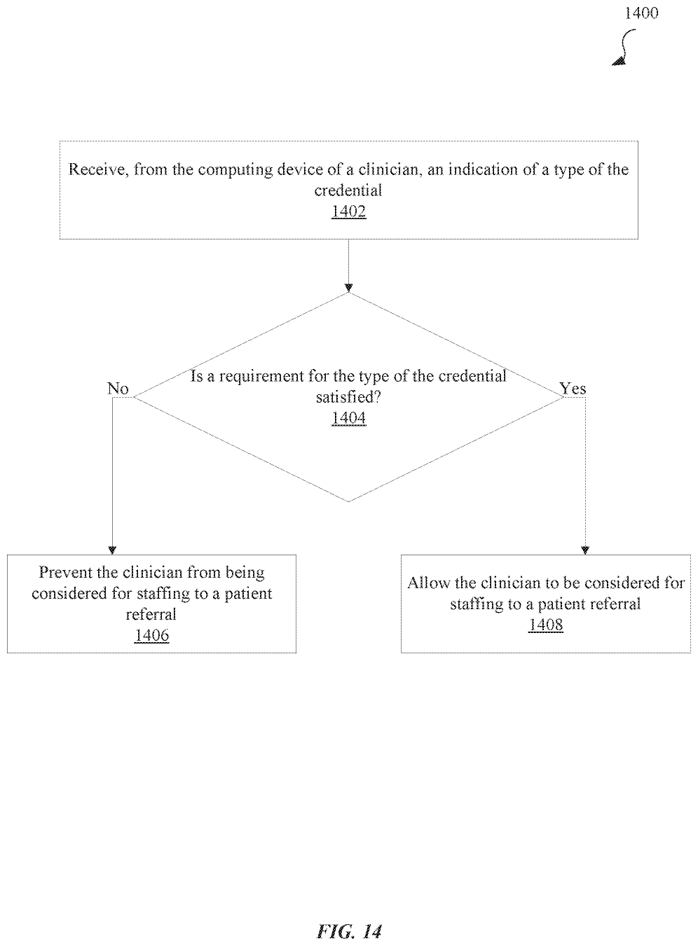

FIG. 14 shows an example method 1400 for determining whether requirements pertaining to a credential issued to a Clinician are satisfied, in accordance with at least some embodiments. Method 1400 includes operations performed by processing devices of the central services system 300 of FIG. 3. In some embodiments, one or more operations of the method 1400 are implemented in computer instructions executable by a processing device of the central services system 300. The method 1400 may be performed in the same or a similar manner as described above in regard to method 700 of FIG. 7.

At operation 1402, the processing device may receive, from the computing device 324-4 of a Clinician, an indication of a type of the credential. The type of credential may pertain to healthcare. At operation 1404, the processing device may determine if a requirement for the type of credential is satisfied. The requirement may include drug screening, certain spoken language, no criminal history, or the like. If the requirement is not satisfied, then at operation 1406, the processing device may prevent the Clinician from being considered for staffing to a patient referral. If the requirement is satisfied, then at operation 1408, the processing device may allow the Clinician to be considered for staffing to a patient referral.

### Real-Time Communication and Data Shielding

There are network bottlenecks in the healthcare industry that delay and/or prevent communications between entities in an efficient and direct manner. For example, as discussed above, a Clinician may need advice on a condition of a patient from the Doctor who performed a surgery on the patient but the Clinician may not have a way to reach the Doctor in real-time or near real-time. The Clinician may call the office where the Doctor works and may reach an assistant who takes a message or the Clinician may have to leave a voicemail. It may take an undesirable amount of time for the Doctor to reply. Also, the message may be altered when delivered to the Doctor due to an assistant failing to take accurate notes. Further, it may be helpful for the Doctor to see an image and/or video of the patient to help make a decision on the severity of the condition on which the Clinician is seeking advice. Conventional communication mechanisms lack the ability to provide such messages with images and/or video directly to a Doctor to enable real-time or near real-time feedback and/or alteration of a treatment plan of the patient.

According to some embodiments of the present disclosure, the central services system 300 (e.g., the data exchange engine 302) may provide a communication mechanism to enable direct real-time or near real-time messaging between entities in the healthcare industry. Communication mappings between the entities may be dynamically created to enable communication between the entities. For example, when patient therapy data for a patient is downloaded by the central services system 300, the data exchange engine 302 may identify a correlation between the identity of the patient and an identity of a Doctor that performed surgery on the patient. After a Clinician is assigned to provide the in-home therapy, the data exchange engine 302 may identify a correlation between the identity of the patient and an identity of the Clinician assigned to provide the in-home therapy. Based on the Clinician and the Doctor being correlated with the patient, the data exchange engine 302 may create a communication mapping between the identity of the Doctor and the identity of the Clinician. Using the communication mapping, the data exchange engine 302 may provide real-time or near real-time messaging between a computing device 324-5 of the Doctor and a computing device 324-4 of the Clinician. Other communication mappings may be created to enable direct real-time or near real-time communication between other entities (e.g., between a Clinician and a patient, between a patient and a Doctor, between a Clinician and a Scheduler, etc.), thereby creating an improved communication network in the healthcare industry. In some embodiments, the efficiency and/or accuracy of data exchange between the computing devices may be improved using the disclosed techniques. In some embodiments, the real-time or near real-time messaging may be chat messaging, text messaging, electronic mail messaging, voice messaging, or the like.

In addition, different EMR platforms 314 may require different organizational structures for data to be processed by and/or stored at the EMR platforms 314. The data exchange engine 302 may enable receiving patient therapy data in one organizational structure from a computing device 324-4 of a Clinician using the Clinician application 318, for example, and creating second patient therapy data in another organizational structure of the destination EMR platform(s) 314. In this way, the data exchange engine 302 enables the central services system 300 to be EMR platform 314 agnostic by being capable of complying with any data organizational structure specifications of the EMR platforms 314. The data exchange engine 302 may call an application programming interface that maps the patient therapy data into the organizational structure(s) of the EMR platform(s) 314. In some embodiments, an artificial intelligence engine may perform the mapping of the patient therapy data into different organizational structures of EMR platforms 314 using one or more machine learning models.

FIG. 15 shows, in block diagram form, an example workflow for real-time or near real-time communication between a Physician and a Clinician, in accordance with at least some embodiments. As depicted, the central services system 300 may electronically download patient data 1500 for a patient from the EMR platform 314. The patient data 1500 may include any suitable information, such as an identity (John Smith) of the patient, an identity (Dr. Jane Doe) of a Physician that provided healthcare to the patient, a discipline of therapy to be provided to the patient, and so forth. The patient therapy data may be stored in the database 308 (e.g., after manipulation of data organizational structure). The central services system 300 (e.g., the data exchange engine 302) may identify a correlation 1502 between the identity of the Physician and the identity of the patient. As discussed herein, the Clinician selection engine 310 may select and assign a Clinician to provide in-home therapy to the patient. An identity (Sally Jones) of the assigned Clinician may be stored in the database 308. The central services system 300 (e.g., the data exchange engine 302) may identify a correlation 1504 between the identity of the patient and the identity of the Clinician assigned to provide the in-home therapy to the patient. The data exchange engine 302 may generate, based on the correlations 1502 and 1504, a communication mapping 1506 between the identity (Dr. Jane Doe) of the Physician and the identity (Sally Jones) of the Clinician. The communication mapping 1506 may specify that accounts associated with the identities of the Physician and Clinician are enabled to provide messages to each other via the Ortho application 320 and the Clinician application 318 when the users are logged into the respective applications with their accounts. The communication mapping 1506 may be stored in the database 308. In some embodiments, when the communication mapping 1506 is generated, an identity of the Physician may appear as a contact in the Clinician application 318 running on the computing device 324-4 of the Clinician, and an identity of the Clinician may appear as a contact in the Ortho application 320 running on the computing device 324-5 of the Physician.

In some embodiments, certain data may be shielded/prevented from being provided to the Clinician application 318 and/or the Ortho application 320. For example, a phone number of the Physician may be prevented from being provided to the Clinician application 318. Instead, just the identity of the Physician may be provided, and the central services system 300 may store the phone number of the Physician in the database 308. When the Clinician wants to send a message to the Physician, the Clinician application 318 may transmit the message to the central services system 300, and the central services system 300 may use the identity of the Physician to lookup the Physician's phone number to transmit the message to the computing device 324-5 of the Physician. In some embodiments, the phone number of the Physician may be provided to the Clinician application 318 but the phone number may not be exposed to the Clinician. For example, the phone number of the Physician may be encrypted and stored on the computing device 324-4 of the Clinician. The Clinician application 318 may expose the identity of the Physician and allow sending messages directly to the computing device 324-5 of the Physician using the phone number that is stored.

The central services system 300 (e.g., the data exchange engine 302) may provide, using the communication mapping, real-time or near real-time messaging between the computing device 324-4 of the Clinician and the computing device 324-5 of the Physician. For example, in some embodiments a chat messaging service may be included in the Clinician application 318 and the Ortho application 320 that enables the real-time or near real-time communication between the devices 324-4 and 324-5 directly and/or via the central services system 300. The Clinician logged into his account may compose a first message 1508 that includes text and/or an attachment 1509. The Clinician may be providing the in-home therapy to the patient at the residence of the patient and decide to ask the Physician a question regarding a condition of the patient with which the Clinician is concerned. The Clinician may select the identity of the Physician as the recipient of the first message 1508. The text may state “Dr. Jane Doe, does this look infected?” The attachment 1509 may include an image and/or video of a body part of the patient that the Clinician thinks may be infected. The first message 1508 including the attachment 1509 may comply with certain statutorily regulated guidelines by being encrypted using end-to-end encryption, for example.

The first message 1508 may be sent to the computing device 324-5 of the Physician specified in the first message 1508 directly or via the central services system 300. In some embodiments, the first message 1508 may be stored in an encrypted state in the database 308 of the central services system 300. The first message 1508 and the attachment 1509 may be presented in a user interface of the Ortho application 320 on a display of the computing device 324-5. The Physician may read the message 1508 and view the attachment 1509 and compose a reply message (second message 1510), that includes text “Sally Jones, no that is OK.” The second message 1510 may be transmitted in real-time or near real-time (e.g., while the Clinician is still providing in-home therapy to the patient) to the computing device 324-4 of the Clinician directly or via the central services system 300.

In some embodiments, the Physician may assign communication privileges to an assistant. For example, the Physician may choose certain people, who also have access to the Ortho application 320, to view messages directed toward the Physician. This may aid in situations where the Physician is away from their computing device 324-5 by allowing the assistant to view the message and try to contact the Physician. As described further below, other communication mappings may be created to enable additional entities in the healthcare industry to directly communicate in real-time or near real-time.

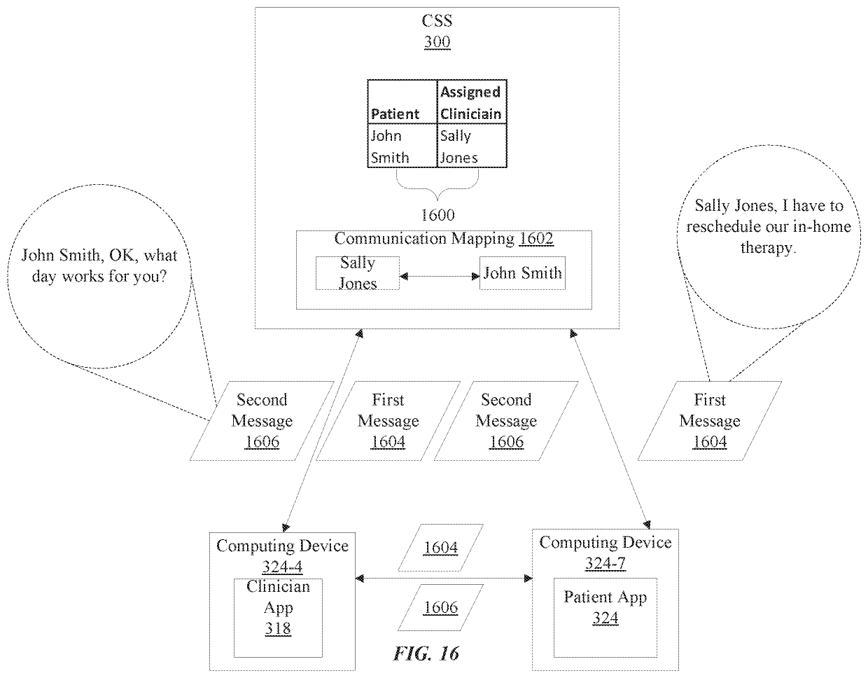

FIG. 16 shows, in block diagram form, an example workflow for real-time or near real-time communication between a patient and a Clinician, in accordance with at least some embodiments. As depicted, the central services system 300 may identify a correlation 1600 between an identity (John Smith) of a patient and an identity (Sally Jones) of a Clinician that is assigned to provide in-home therapy to the patient. The correlation 1600 may be identified in the database 308 that stores the assignment of the identity of the Clinician to the identity of the patient. The central services system 300 may generate a communication mapping 1602 between the identity of the patient and the identity of the Clinician. The communication mapping 1602 may be stored in the database 308. Using the communication mapping 1602, real-time or near real-time messaging may be provided between the computing device 324-7 of the patient and the computing device 324-4 of the Clinician. In some embodiments, a chat messaging service may be included in the patient application 324 and the Clinician application 318 to enable chat messages to be transmitted between the computing devices 324-7 and 324-4. In some embodiments, when the communication mapping 1602 is generated, an identity of the Clinician may appear as a contact in the patient application 324 running on the computing device 324-7 of the patient, and an identity of the patient may appear as a contact in the Clinician application 318 running on the computing device 324-4 of the Clinician.

For example, the patient may decide to contact their assigned Clinician for any suitable reason (e.g., question regarding the schedule for in-home therapy). The patient may be logged into the patient application 324 running on the computing device 324-7 and may compose a first message 1604 directed to the identity (Sally Jones) of the Clinician. The first message 1604 may include text “Sally Jones, I have to reschedule our in-home therapy.” In some embodiments, the first message 1604 may include an attachment. The first message 1604 may be encrypted. The computing device 324-7 may transmit the first message 1604 to the computing device 324-4 of the Clinician directly or via the central services system 300.

The Clinician application 318 running on the computing device 324-4 may present the first message 1604 on a user interface. The Clinician may select to reply to the first message 1604 and may compose a second message 1606 directed to the identity of the patient. The second message 1606 may include text “John Smith, OK, what day works for you?” The second message 1606 may be encrypted. The second message 1606 may be transmitted by the computing device 324-4 to the computing device 324-7 of the patient and may be presented on a user interface of the patient application 324.

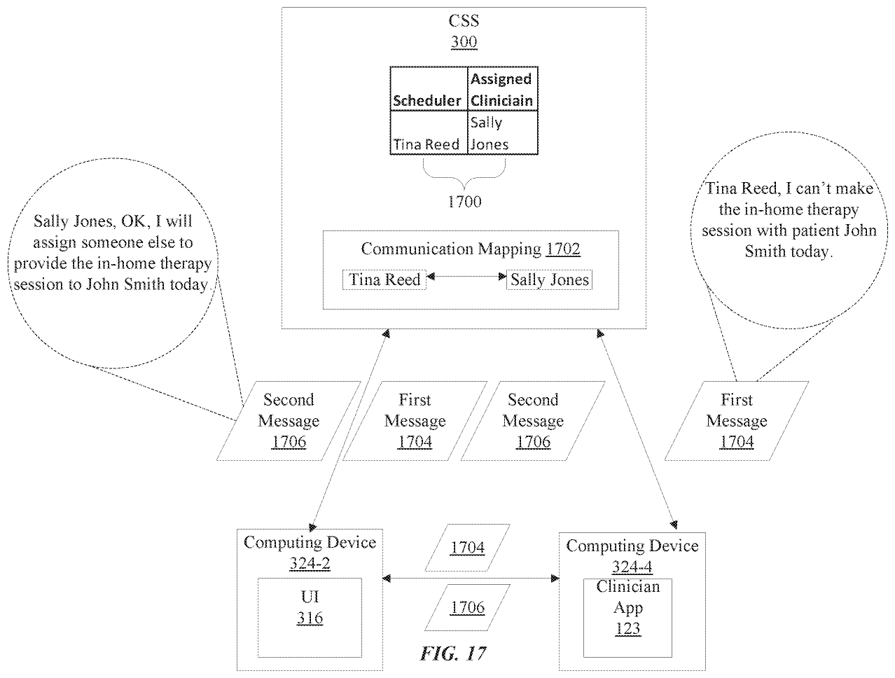

FIG. 17 shows, in block diagram form, an example workflow for real-time or near real-time communication between a Scheduler and a Clinician, in accordance with at least some embodiments. As depicted, the central services system 300 may identify a correlation 1700 between an identity (Tina Reed) of a Scheduler and an identity (Sally Jones) of a Clinician, where the Scheduler assigned the Clinician to provide the in-home therapy to the patient. The correlation 1700 may be identified in the database 308 that stores the identity of the Scheduler who assigned the Clinician to provide the in-home therapy to the patient. The central services system 300 may generate a communication mapping 1702 between the identity of the Scheduler and the identity of the Clinician. The communication mapping 1702 may be stored in the database 308. Using the communication mapping 1702, real-time or near real-time messaging may be provided between the computing device 324-2 of the Scheduler and the computing device 324-4 of the Clinician. In some embodiments, a chat messaging service may be included in the user interface 316 and the Clinician application 318 to enable chat messages to be transmitted between the computing devices 324-2 and 324-4. In some embodiments, when the communication mapping 1702 is generated, an identity of the Clinician may appear as a contact in the user interface 316 running on the computing device 324-2 of the Scheduler, and an identity of the patient may appear as a contact in the Clinician application 318 running on the computing device 324-4 of the Clinician.

For example, the Clinician may decide to contact the Scheduler for any suitable reason (e.g., question regarding the schedule for in-home therapy). The Clinician may be logged into the Clinician application 318 running on the computing device 324-4 and may compose a first message 1704 directed to the identity (Tina Reed) of the Scheduler. The first message 1704 may include text “Tina Reed, I can't make the in-home therapy session with patient John Smith today.” In some embodiments, the first message 1704 may include an attachment. The first message 1704 may be encrypted. The computing device 324-4 may transmit the first message 1704 to the computing device 324-2 of the Scheduler directly or via the central services system 300.

The user interface 316 running on the computing device 324-2 may present the first message 1704. The Scheduler may select to reply to the first message 1704 and may compose a second message 1706 directed to the identity of the Clinician. The second message 1706 may include text “Sally Jones, OK, I will assign someone else to provide the in-home therapy session to John Smith today.” The second message 1706 may be encrypted. The second message 1706 may be transmitted by the computing device 324-2 to the computing device 324-4 of the Clinician and may be presented on a user interface of the Clinician application 318.

Other communication mappings may be created between various entities in the healthcare industry. For example, a communication mapping may be created between an identity of a patient and an identity of a Physician that provided healthcare to the patient to enable the patient and the Physician to communicate in real-time or near real-time. In another example, a communication mapping may be created between an identity of a patient and an insurance provider to enable real-time or near real-time communication between the patient and the insurance provider.

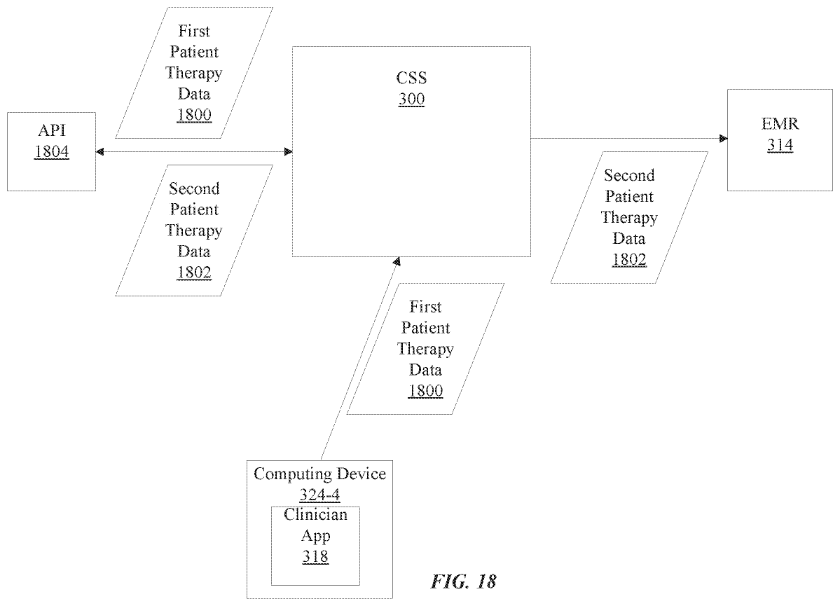

FIG. 18 shows, in block diagram form, an example workflow for creating patient therapy data having an organizational structure of an EMR platform 314, in accordance with at least some embodiments. A Clinician may provide in-home therapy to a patient and enter patient notes regarding the in-therapy session into the Clinician application 318 running on the computing device 324-4. The patient notes may include an outcome of the in-home therapy session, a range of motion of the patient, vital statistics of the patient, a date and time of the in-home therapy session, and so forth. The patient notes may be referred to as a first patient therapy data 1800. The first patient therapy data 1800 may be transmitted to the central services system 300 by the computing device 324-4. The first patient therapy data 1800 may be encrypted. The first patient therapy data 1800 may have a first organizational structure that is specific to a component (e.g., database 308) of the central services system 300. The central services system 300 may store the first patient therapy data 1800 in the database 308.

The central services system 300 may create a second patient therapy data 1802 that has a second organizational structure specific to an EMR platform 314. In some embodiments, the EMR platform 314 may be associated with the Home Health Agency with which the Clinician is a member. In some embodiments, the central services system 300 may call an application programming interface (API) 1804 to map the fields of the first patient therapy data in the first organizational structure to the fields of the second patient therapy data in the second organizational structure. Although the API 1804 is depicted external to the central services system 300, the API 1804 may be an integral component of the central services system 300 in some embodiments. In addition, the central services system 300 may use an artificial intelligence engine including one or more machine learning models to create the second patient therapy data 1802. The machine learning models may be trained with training data that identifies the mappings of fields from the first organizational structure to the second organizational structure. The second patient therapy data 1802 may be transmitted to the EMR platform 314 for processing and/or storage.

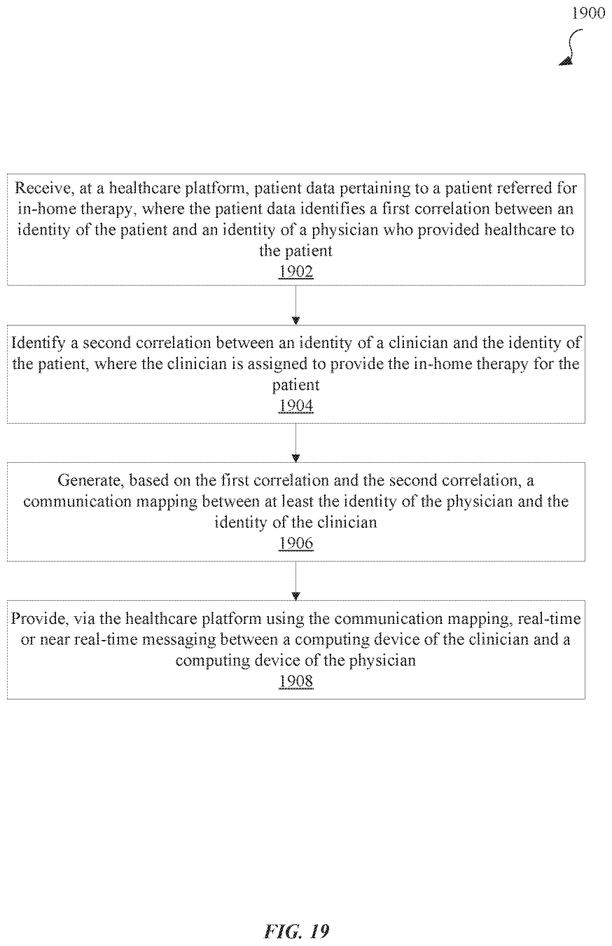

FIG. 19 shows an example method 1900 for using a communication mapping to provide real-time or near real-time messaging between computing devices, in accordance with at least some embodiments. Method 1900 includes operations performed by processing devices of the central services system 300 of FIG. 3. In some embodiments, one or more operations of the method 1900 are implemented in computer instructions executable by a processing device of the central services system 300. The method 1900 may be performed in the same or a similar manner as described above in regard to method 700 of FIG. 7.

At operation 1902, the processing device may receive, at a healthcare platform (e.g., central services system 300), patient data pertaining to a patient referred for in-home therapy, where the patient data identifies a first correlation between an identity of the patient and an identity of a physician who provided healthcare to the patient. For example, the healthcare platform may electronically download the patient data from an EMR platform 314 after a patient referral is received by a Home Health Agency, for example, and the patient data is entered into the EMR platform 314 based on the patient referral. The patient data may specify that the identity of the Physician that provided the healthcare to the patient.

At operation 1904, the processing device may identify a second correlation between an identity of a Clinician and the identity of the patient, where the Clinician is assigned to provide the in-home therapy for the patient. As described above, the Clinician may be automatically assigned to provide the in-home therapy for the patient, or a Scheduler may select and assign the Clinician to provide the in-home therapy for the patient. The correlation may be identified based on data stored in the database 308, where the data includes the identity of the patient and the identity of the assigned Clinician.

At operation 1906, the processing device may generate, based on the first correlation and the second correlation, a communication mapping between at least the identity of the physician and the identity of the Clinician. In some embodiments, the communication mapping may specify that the accounts of the Clinician and the Physician have permission to communicate messages with each other using the Clinician application 318 and the Ortho application 320. The communication mapping may be stored in the database 308. Further, a notification may be presented by the Clinician application 318 on the computing device 324-4 of the Clinician, where the notification indicates communication with the Physician is now enabled. Further, the identity of the Physician may be added as a contact in the Clinician application 318. A notification may be presented by the Ortho application 320 on the computing device 324-5 of the Physician, where the notification indicates communication with the Clinician is now enabled. Further, the identity of the Clinician may be added as a contact in the Ortho application 320.

At operation 1908, the processing device may provide, using the communication mapping, real-time or near real-time messaging between the computing device 324-4 of the Clinician and the computing device 324-5 of the Physician. In some embodiments, the messaging may be chat messaging. In some embodiments, the processing device may prevent a phone number associated with the Physician from being provided to the computing device 324-4 of the Clinician. In some embodiments, the phone number of the Physician may be provided to the computing device 324-4 of the Clinician but the phone number may be in an encrypted state and may not be exposed on a user interface of the Clinician application 318.

Other communication mappings may be generated and used to provide real-time or near real-time messaging. For example, the processing device may generate, based on the first correlation between the identity of the Clinician and the identity of the patient, a communication mapping between the identity of the Clinician and the identity of the patient. The processing device may provide, using the communication mapping, real-time or near real-time messaging between the computing device 324-4 of the Clinician and the computing device 324-7 of the patient.

In some embodiments, the processing device may identify a third correlation between the identity of the Clinician and an identity of a Scheduler that assigned the Clinician to provide the in-home therapy for the patient. The processing device may generate, based on the third correlation, a communication mapping between the identity of the Clinician and the identity of the Scheduler. The processing device may provide, using the communication mapping, real-time or near real-time messaging between the computing device 324-4 of the Clinician and the computing device 324-2 of the Scheduler.

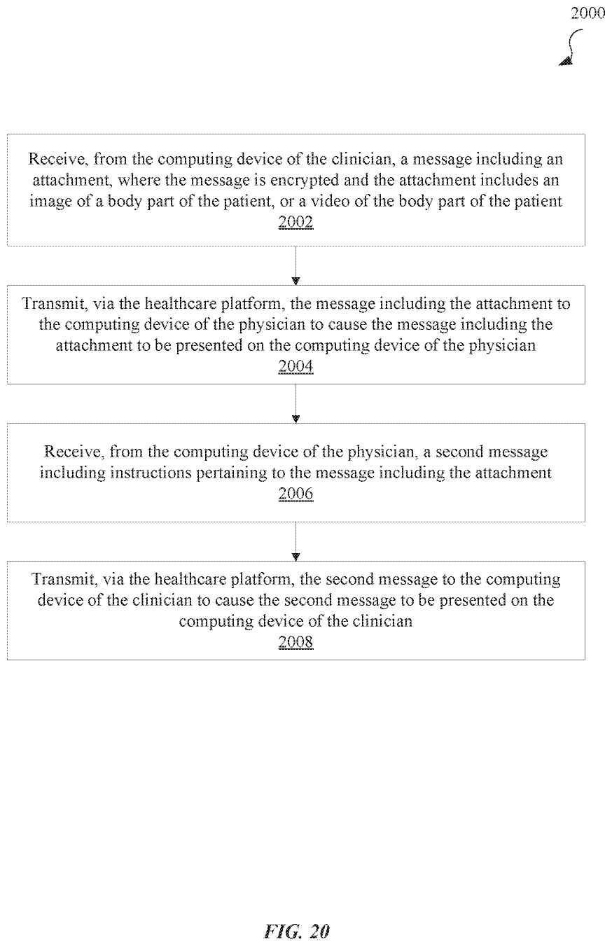

FIG. 20 shows an example method 2000 for transmitting encrypted messages with attachments between computing devices, in accordance with at least some embodiments. Method 2000 includes operations performed by processing devices of the central services system 300 of FIG. 3. In some embodiments, one or more operations of the method 2000 are implemented in computer instructions executable by a processing device of the central services system 300. The method 2000 may be performed in the same or a similar manner as described above in regard to method 700 of FIG. 7.

At operation 2002, the processing device may receive, from the computing device 324-4 of the Clinician, a message including an attachment. In some embodiments, the message may be transmitted from the computing device 324-4 of the Clinician directly to the computing device 324-5 of the Physician. The message may be encrypted and the attachment may include an image of a body part of the patient, or a video of the body part of the patient. End-to-end encryption may be used to encrypt the message. In some embodiments, public/private key encryption may be used. The computing device 324-4 of the Clinician may transmit a public key to the central services system 300 and/or a computing device 324-5 of the Physician and may sign the message with a private key. The central services system 300 and/or the computing device 324-5 of the Physician may decrypt the message using the public key.

At operation 2004, the processing device may transmit the message including the attachment to the computing device 324-5 of the Physician to cause the message including the attachment to be presented on the computing device 324-5 of the Physician (via the Ortho application 320). The Physician may use the Ortho application 320 to compose a second message to the Clinician. The computing device 324-5 of the Physician may transmit the second message to the central services system 300 and/or the computing device 324-4 of the Clinician.

At operation 2006, the processing device may receive, from the computing device 324-5 of the Physician, the second message including instructions pertaining to the message including the attachment. The instructions may include any suitable reply, such as instructing the Clinician to apply an ointment to the patient, provide medication to the patient, do nothing, etc.

At operation 2008, the processing device may transmit the second message to the computing device 324-4 of the Clinician to cause the second message to be presented on the computing device 324-4 of the Clinician. The second message may be received and presented in real-time or near real-time. For example, the second message may be received and presented while the Clinician is still providing the in-home therapy to the patient. Such direct and efficient communication may greatly enhance the outcome of in-home therapy sessions.

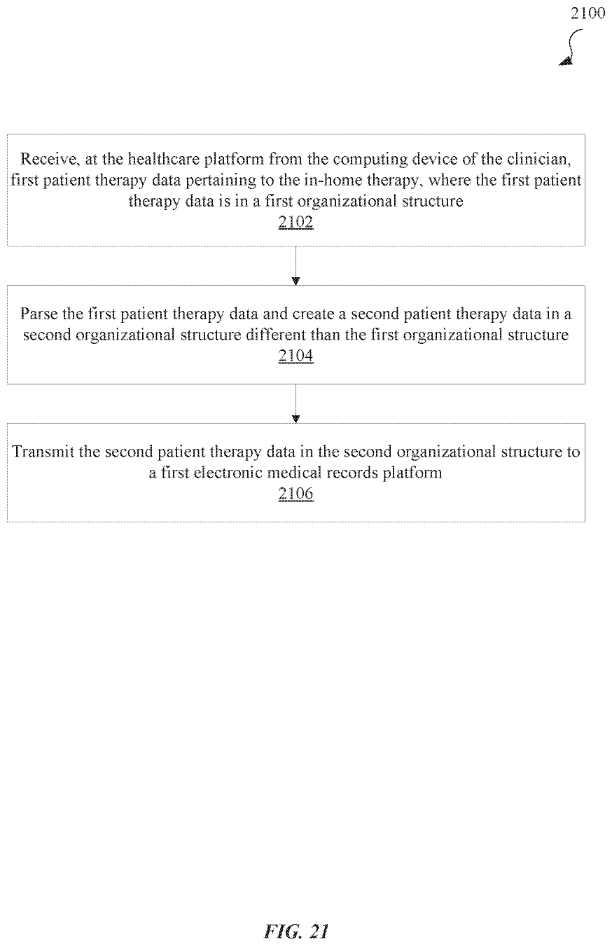

FIG. 21 shows an example method 2100 for creating patient therapy data having an organizational structure of an EMR platform, in accordance with at least some embodiments. Method 2100 includes operations performed by processing devices of the central services system 300 of FIG. 3. In some embodiments, one or more operations of the method 2100 are implemented in computer instructions executable by a processing device of the central services system 300. The method 2100 may be performed in the same or a similar manner as described above in regard to method 700 of FIG. 7.

At operation 2102, the processing device may receive, from the computing device 324-4 of the Clinician, first patient therapy data pertaining to in-home therapy, where the first patient therapy data is in a first organizational structure. The first patient therapy data may be entered by the Clinician using the Clinician application 318 before, during, or after the in-home therapy session. For example, the first patient therapy data may include an outcome of the in-home therapy session.

At operation 2104, the processing device may parse the first patient therapy data and create a second patient therapy data in a second organizational structure different than the first organizational structure. The second organizational structure may be specific to a first EMR platform 314 associated with a Home Health Agency, for example, of which the Clinician is a member. At operation 2106, the processing device may transmit the second patient therapy data in the second organizational structure to the first EMR platform 314.

In some embodiments, parsing the first patient therapy and creating the second patient therapy data, and transmitting the second patient therapy data, further comprises invoking an application programming interface (API) that maps the first patient therapy data in the first organizational structure into the second patient therapy data in the second organizational structure, and transmitting the second patient therapy data to the first EMR platform using a first communication scheme of the first EMR platform.

In some embodiments, the processing device may parse the first patient therapy data and create a third patient therapy data in a third organizational structure different than the first or second organizational structure. The processing device may transmit the third patient therapy data in the third organizational structure to a second EMR platform 314 different than the first EMR platform 314. In some embodiments, parsing the first patient therapy data and creating the second patient therapy data, and transmitting the second patient therapy data, further comprises invoking an application programming interface (API) that maps the first patient therapy data in the first organizational structure into the second patient therapy data in the second organizational structure, and transmitting the second patient therapy data to the first EMR platform using a first communication scheme of the first EMR platform. In some embodiments, parsing the first patient therapy data and creating the third patient therapy data, and transmitting the third patient therapy data, further comprises invoking a second API that maps the first patient therapy data in the first organizational structure into the third patient therapy data in the third organizational structure, and transmitting the third patient therapy data to the second EMR platform using a second communication scheme of the second EMR platform.

### Data Analytics of Outcomes and Pay-for-Performance Metrics

There is a wealth of data that may be obtained by the central services system 300 described herein. Data pertaining to Clinicians, Doctors, patients, therapy sessions, medical procedures, and so forth, may be aggregated in the database 308. In some embodiments, the analytics engine 304 of the central services system 300 may analyze the data and generate new datasets and/or provide targeted insights that enable real-time actions to be performed. For example, the analytics engine 304 may analyze patient notes from therapy sessions and determine outcomes for patients that went through the therapy sessions. The analytics engine 304 may group patients based on the outcomes to create a group of favorable outcomes and a group of unfavorable outcomes. Further, the analytics engine 304 may determine at least one root cause of favorable outcomes, and suggest changes to future therapy sessions based on the at least one root cause of favorable outcomes. Relatedly, the analytics engine may assign pay-for-performance values or metrics to each of the plurality of Doctors, each of the plurality of Clinicians, and/or each of the plurality of therapy protocols. Further still, the analytics engine 304 may predict future outcomes of future patients based on the future patient's doctor, Clinician, therapy protocols, and/or medical device vendors. Thus, the analytics engine 304 creates data that never previously existed across a variety of doctors, vendors, patients, HHA, Staffing Companies, and EMR platforms, and thus improves another technological field.

The analytics engine 304 may generate various reports that are presented on the user interface 316 of the computing device 324-4 of the Scheduler. The reports may include insights gleaned from analyzing the data, where the insights provide actionable items in real-time or near real-time that enable the Scheduler to take an action. Thus, the user interface 316 may function as an action center for the central services system 300. For example, one report may provide an indication that a set of Clinicians are not able to satisfy a certain type of therapy session, and thus, a notification may be presented that recommends hiring another Clinician trained in that type of therapy session. Another report may indicate that the availability of a set of Clinicians to handle a predicted amount of referrals in the future is not sufficient, and a recommendation may indicate hiring additional Clinicians. Further, a report may present indications when Clinicians cannot make scheduled therapy sessions and enable scheduling another matched Clinician to provide the scheduled therapy session.

FIG. 22 shows, in block diagram form, an example workflow for determining a root cause of a therapy session having a favorable outcome and providing a recommendation, in accordance with at least some embodiments. As depicted, patient notes 2200 may be entered by a Clinician using the Clinician application 318 running on the computing device 324-4. The patient notes 2200 may include a set of data. The patient notes 2200 may include an identity of the patient, an identity of a Doctor that provided healthcare to the patient, an identity of a therapy protocol, an identity of a Clinician that provided the therapy session, an identity of a medical device vendor that provides a medical device used during the healthcare and/or the therapy session, an outcome associated with the patient, and the like. The patient notes 2200 may be transmitted to the central services system 300 in any suitable data format (e.g., extensible markup language (XML), JavaScript Object Notation (JSON), etc.). Numerous patient notes 2200 may be received from numerous computing devices 324-4, resulting in a set of patient notes.

The central services system 300 (e.g., the analytics engine 304) may detect an outcome for each patient of the numerous patients. In some embodiments, the outcome may be explicitly provided in the set of data representing the patient notes 2200. For example, a Clinician and/or a Doctor may provide a score, a grade, a description, or the like for an outcome for the patient. In some embodiments a machine learning model 2202 may be used to detect the outcome. The machine learning model 2202 may be included as part of an artificial intelligence engine. Although just one machine learning model 2202 is depicted, it should be understood that any suitable number of machine learning models 2202 may be used to perform one or more operations described herein. The machine learning model 2202 may be trained with training data that includes patient notes having certain criteria (e.g., a certain Doctor, a certain Clinician, a certain therapy protocol, etc.) and an indicated outcome that results based on those certain criteria.

The central services system 300 may group the numerous patients based on the set of outcomes to create a group of favorable outcomes and a group of unfavorable outcomes. Further, the central services system 300 may analyze an underlying cause in a difference between the group of unfavorable outcomes and the group of favorable outcomes to determine a root cause of favorable outcomes. The central services system 300 may determine a recommendation to modify a future therapy session based on the root cause of favorable outcomes. The machine learning model 2202 may be trained to group the patients in the group of favorable outcomes and the group of unfavorable outcomes, analyze the differences between the group of favorable outcomes and the group of unfavorable outcomes to output the root cause of favorable outcomes, and/or recommend a modification to future therapy sessions based on the root cause.

The central services system 300 may transmit a notification 2204 including the root cause and/or the recommendation to the computing device 324-4 of the Clinician associated with the patient and/or the computing device 324-5 of the Doctor associated with the patient. The root cause and the recommendation for future therapy sessions may be presented on a display of the computing devices 324-4 and/or 324-5.

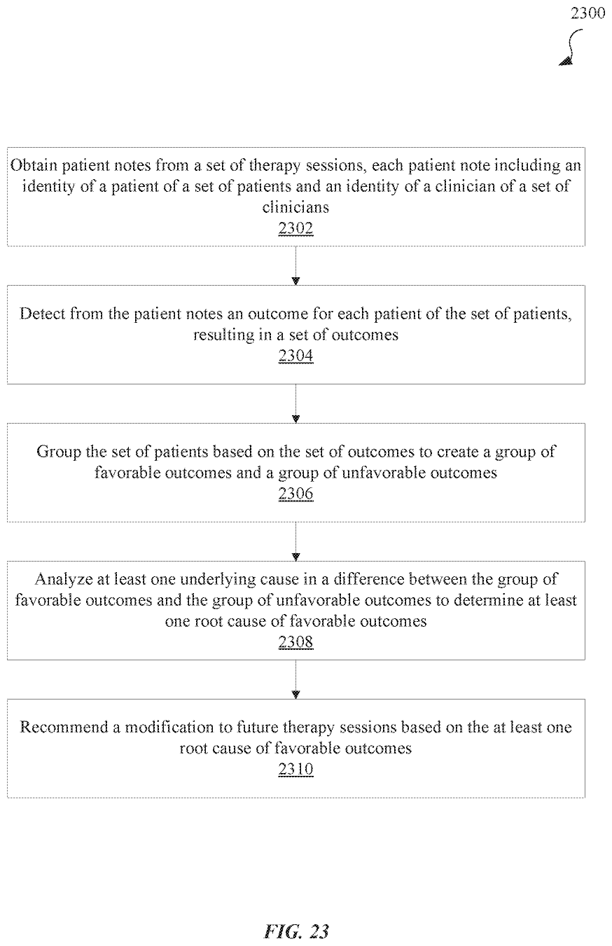

FIG. 23 shows an example method 2300 for determining a root cause of a therapy session having a favorable outcome and providing a recommendation, in accordance with at least some embodiments. Method 2300 includes operations performed by processing devices of the central services system 300 of FIG. 3. In some embodiments, one or more operations of the method 2300 are implemented in computer instructions executable by a processing device of the central services system 300. The method 2300 may be performed in the same or a similar manner as described above in regard to method 700 of FIG. 7.

At operation 2302, the processing device may obtain patient notes from a set of therapy sessions. Each patient note may include an identity of a patient of a set of patients and an identity of a Clinician of a set of Clinicians, where the Clinician provided the set of therapy sessions.

At operation 2304, the processing device may detect from the patient notes an outcome for each patient of the set of patients, resulting in a set of outcomes. The outcome may be included as a field of text in the patient notes and the processing device. The text of the outcomes may be a grade, a score, a description of the outcome, or the like associated with the outcome for the patient. For example, the outcome may state “Full recovery.” As described above, the outcome may be detected by the machine learning model.

At operation 2306, the processing device may group the set of patients based on the set of outcomes to create a group of favorable outcomes and a group of unfavorable outcomes. In some embodiments, the processing device may analyze the outcomes in relation to a lookup table that indicates whether the outcomes are favorable or unfavorable. The machine learning model may be trained to identify and group the patients based on the set of outcomes to create the group of favorable outcomes and the group of unfavorable outcomes. For example, the machine learning model may be trained with a set of training data that includes patient data having outcomes that are identified as favorable or unfavorable, and the trained machine learning model may be able to label the patient data as favorable or unfavorable based on the outcomes.

At operation 2308, the processing device may analyze at least one underlying cause in a difference between the group of favorable outcomes and the group of unfavorable outcomes to determine at least one root cause of favorable outcomes. The processing device may analyze numerous criteria for the underlying causes. For example, the processing device may look at the therapy protocol used during therapy, the identity of the Doctor that provided healthcare for the patient, the identity of the Clinician that provided the therapy sessions for the patient, a medical device used for the healthcare and/or the therapy sessions, symptoms of the patient, medical history of the patient, a duration of therapy sessions, and so forth. The processing device may compare the criteria from the group of unfavorable outcomes and the group of unfavorable outcomes to determine the difference and to determine the root cause of the favorable outcomes. In some embodiments, the machine learning model may be trained to compare various criteria and identify a root cause of the favorable outcomes.

At operation 2310, the processing device may recommend a modification to future therapy sessions based on the at least one root cause of favorable outcomes. For example, if the root cause for the favorable outcomes is a certain therapy protocol is used instead of a different therapy protocol associated with the unfavorable outcomes, the processing device may recommend using the certain therapy protocol for future therapy sessions. Any suitable recommendation may be provided based on the at least one root cause that is determined. In some embodiments, the machine learning model may be trained to generate and output a recommendation for a modification for future therapy sessions based on the root cause. For example, training data that identifies certain root causes and associated recommendations may be used to train the machine learning model.

In some embodiments, each patient note may include an identity of a Doctor of a set of Doctors, where the Doctor provided healthcare to the patient (e.g., performed surgery on the patient). The processing device may assign, based on the set of outcomes, a Doctor metric to each Doctor to the set of Doctors. The processing device may assign, based on the set of outcomes, a Clinician metric to each Clinician of the set of Clinicians. The processing device may predict a future outcome of a future patient based on a Doctor metric of a selected Doctor from the set of Doctors, and based on a Clinician metric of a selected Clinician of the set of Clinicians.

In some embodiments, each patient note may include an identity of a medical device vendor of a set of medical device vendors, where the medical device vendor provided the medical device used during at least one of the therapy sessions and/or during the healthcare provided by the Doctor. The processing device may assign, based on the set of outcomes, a performance metric to each medical device vendor of the set of medical device vendors. The processing device may select a medical device vendor of the set of medical device vendors as a similar vendor with respect to a future medical device vendor. The processing device may predict a future outcome of a future patient based on the performance metric of the similar vendor.

In some embodiments, each patient note may include an identity of a therapy protocol of a set of therapy protocols, where the therapy protocol may have been assigned by the Doctor that provided the healthcare. The therapy protocol may include a plan of performing a certain number of therapy sessions over a period of time and may specify the type of therapy to be provided during each therapy session and a duration the therapy is to be provided. The processing device may assign, based on the set of outcomes, a performance metric to each therapy protocol of the set of therapy protocols. The processing device may select a therapy protocol of the set of therapy protocols as a similar protocol with respect to a future therapy protocol. The processing device may predict a future outcome of a future patient based on the performance metric of the similar protocol.

In some embodiments, the processing device may predict a number of referrals that will be received in a future time period based on historical information pertaining to referrals received during similar time periods in the past. The processing device may determine a subset of the set of Clinicians that have availability to provide a therapy session in the future time period. The processing device may determine which Clinicians are projected, based on a schedule, to be under-utilized in relation to their overall capacity to work during the future time period. The processing device may provide a list of the subset of Clinicians for presentation on a computing device 324-2 of a Scheduler. The list may rank the under-utilized Clinicians towards the top of the list such that the Scheduler may select the under-utilized Clinicians first. The processing device may determine, based on the list, whether the Clinicians with availability is sufficient to handle the predicted number of referrals and may provide an indication pertaining to the same. If the predicted number of referrals exceeds the availability of the Clinicians to handle the referrals, the Scheduler may determine to hire another Clinician for the future time period. The user interface 316 may provide the list of the subset of Clinicians and/or the indication, which enables the Scheduler to take the appropriate action. To that end, the user interface 316 may function as an action center by generating and/or surfacing actionable data that enables the Scheduler to perform their job more optimally for a company.

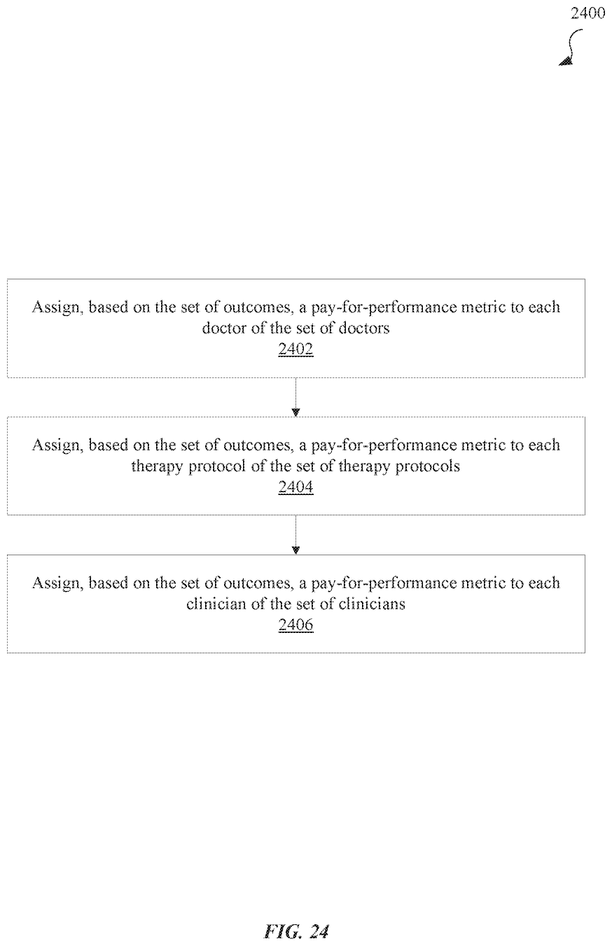

FIG. 24 shows an example method 2400 for assigning pay-for-performance metrics, in accordance with at least some embodiments. Method 2400 includes operations performed by processing devices of the central services system 300 of FIG. 3. In some embodiments, one or more operations of the method 2400 are implemented in computer instructions executable by a processing device of the central services system 300. The method 2400 may be performed in the same or a similar manner as described above in regard to method 700 of FIG. 7.

One or more of the operations of the method 2400 may use the patients notes described above. In some embodiments, each patient note may include an identity of the Doctor of a set of Doctors, where each of the set of patients were provided healthcare by a Doctor in the set of Doctors. In some embodiment, each patient note may include an identity of a therapy protocol of a set of therapy protocols, where the therapy protocol was implemented during a therapy session associated with a respective patient note.

At operation 2402, the processing device may assign, based on the set of outcomes, a pay-for-performance metric to each Doctor of the set of Doctors. The pay-for-performance metric may be a score, a grade, a value, or any suitable metric that represents the quality of the outcomes that result from the healthcare provided by each Doctor. For example, if the outcomes are unfavorable (e.g., a treated condition reoccurs or worsens), the Doctor that provided healthcare to the patients having the unfavorable outcomes may be assigned a pay-for-performance metric that is low. The pay-for-performance metric may be used to evaluate and assign a cost for the Doctor to provide healthcare. In some embodiments, the cost may be determined by an insurance provider using the pay-for-performance metric assigned to the set of Doctors.

At operation 2404, the processing device may assign, based on the set of outcomes, a pay-for-performance metric to each therapy protocol of the set of therapy protocols. The pay-for-performance metric may be a score, a grade, a value, or any suitable metric that represents the quality of the outcomes that result from the therapy protocol being followed. For example, if the outcomes are unfavorable (e.g., a treated condition reoccurs or worsens), the therapy protocol that was used during the therapy sessions for the patients having the unfavorable outcomes may be assigned a pay-for-performance metric that is low. The pay-for-performance metric may be used to evaluate and assign a cost for the therapy protocols. In some embodiments, the cost may be determined by an insurance provider using the pay-for-performance metric assigned to the set of therapy protocols.

At operation 2406, the processing device may assign, based on the set of outcomes, a pay-for-performance metric to each Clinician of the set of Clinicians. The pay-for-performance metric may be a score, a grade, a value, or any suitable metric that represents the quality of the outcomes that result from the Clinician providing the therapy sessions. For example, if the outcomes are unfavorable (e.g., a treated condition reoccurs or worsens), the Clinician that provided the therapy sessions for the patients having the unfavorable outcomes may be assigned a pay-for-performance metric that is low. The pay-for-performance metric may be used to evaluate and assign a cost for using the Clinician. In some embodiments, the cost may be determined by an insurance provider using the pay-for-performance metric assigned to the set of Clinicians.

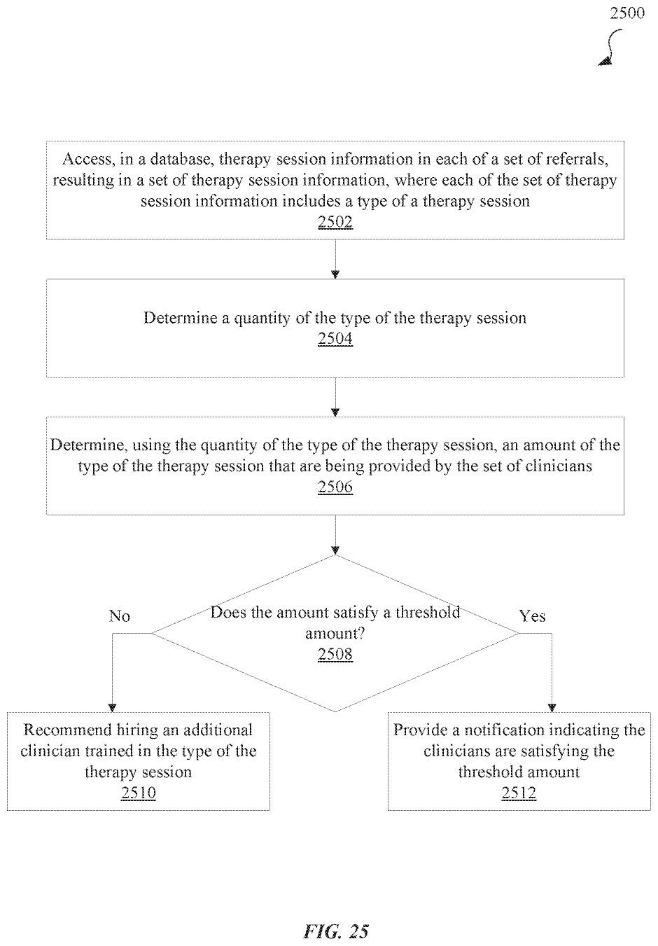

FIG. 25 shows an example method 2500 for determining whether a satisfactory amount of a type of therapy session are being provided by clinicians, in accordance with at least some embodiments. Method 2500 includes operations performed by processing devices of the central services system 300 of FIG. 3. In some embodiments, one or more operations of the method 2500 are implemented in computer instructions executable by a processing device of the central services system 300. The method 2500 may be performed in the same or a similar manner as described above in regard to method 700 of FIG. 7.

At operation 2502, the processing device may access, in a database 308, therapy session information in each of a set of referrals, resulting in a set of therapy session information, where each of the set of therapy session information includes a type (e.g., physical, psychological, wound care, etc.) of a therapy session. The therapy session information may be electronically downloaded by the central services system 300 from one or more EMR platforms 314.

At operation 2504, the processing device may determine a quantity of the type of the therapy session that is specified in the set of referrals. At operation 2506, the processing device may determine, using the quantity of the type of the therapy session, an amount of the type of the therapy session that are being provided by the set of Clinicians. In some embodiments, the amount may be a percentage (e.g., the set of Clinicians are handling 15% of the quantity of the type of therapy sessions specified in the set of referrals). The amount may be any suitable value or portion that indicates how much of the quantity of the type of the therapy sessions are being provided by the set of Clinicians.

At operation 2508, the processing device may determine whether the amount satisfies a threshold amount. The threshold amount may be configurable and may be any suitable amount. For example, in some embodiments, the threshold amount may be 50-100%. If the amount (e.g., 15%) does not satisfy the threshold amount, the processing device may recommend hiring an additional Clinician trained in the type of the therapy session at operation 2510. If the amount does satisfy the threshold amount, the processing device may provide a notification indicating the set of Clinicians are satisfying the threshold amount at operation 2512. The notifications may be presented on the user interface 316 of the computing device 324-2 of a Scheduler, for example. The notifications may enable the Scheduler to take action based on the insight provided, thereby allowing the user interface 316 to function as an action center.

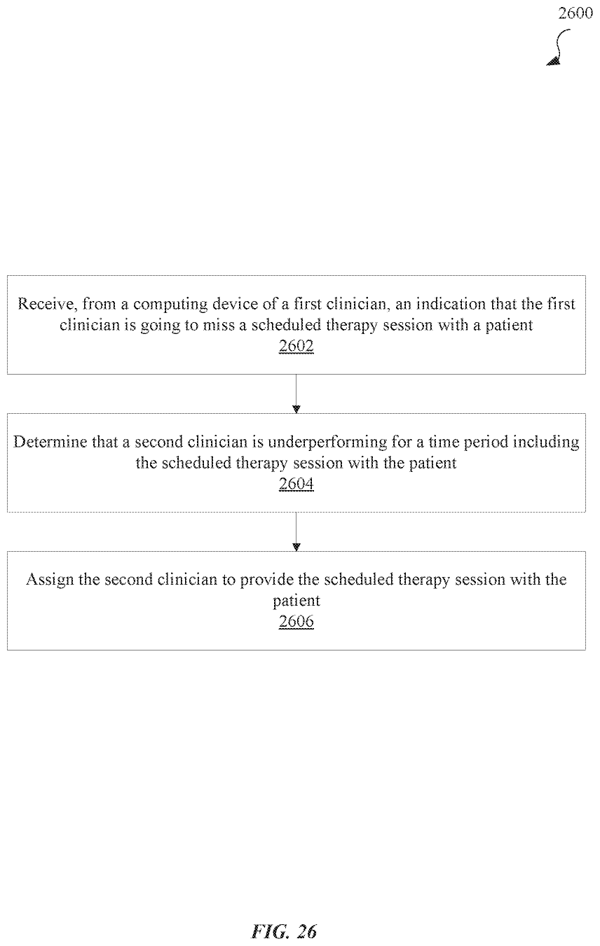

FIG. 26 shows an example method 2600 for assigning a new Clinician to provide in-home therapy when another Clinician is unavailable, in accordance with at least some embodiments. Method 2600 includes operations performed by processing devices of the central services system 300 of FIG. 3. In some embodiments, one or more operations of the method 2600 are implemented in computer instructions executable by a processing device of the central services system 300. The method 2600 may be performed in the same or a similar manner as described above in regard to method 700 of FIG. 7.

At operation 2602, the processing device may receive, from the computing device 324-4 of a first Clinician, an indication that the first Clinician is going to miss a scheduled therapy session with a patient. For example, the Clinician may have a conflict that cannot be rescheduled and may provide the indication via their computing device 324-4 running the Clinician application 318. In another example, the patient may cancel the scheduled therapy session and the Clinician may provide the indication that the scheduled therapy session is going to be missed.

At operation 2604, the processing device may determine that a second Clinician is underperforming for a time period including the scheduled therapy session with the patient. The processing device may use the techniques described herein to find an appropriate matching Clinician to provide the scheduled therapy session with the patient. The processing device may compare the performance of the Clinicians by analyzing their booked therapy sessions (utilization) in relation to their overall capacity to work for the time period. The processing device may rank Clinicians that are underperforming higher in a list of the Clinicians when determining which Clinician to select.

At operation 2606, the processing device may assign the second Clinician to provide the scheduled therapy session with the patient. In this way, instead of allowing the scheduled therapy session to be missed, the processing device may identify a backup Clinician that is matched to provide the scheduled therapy session for the patient and may take action in real-time by assigning the second Clinician to provide the scheduled therapy session for the patient.

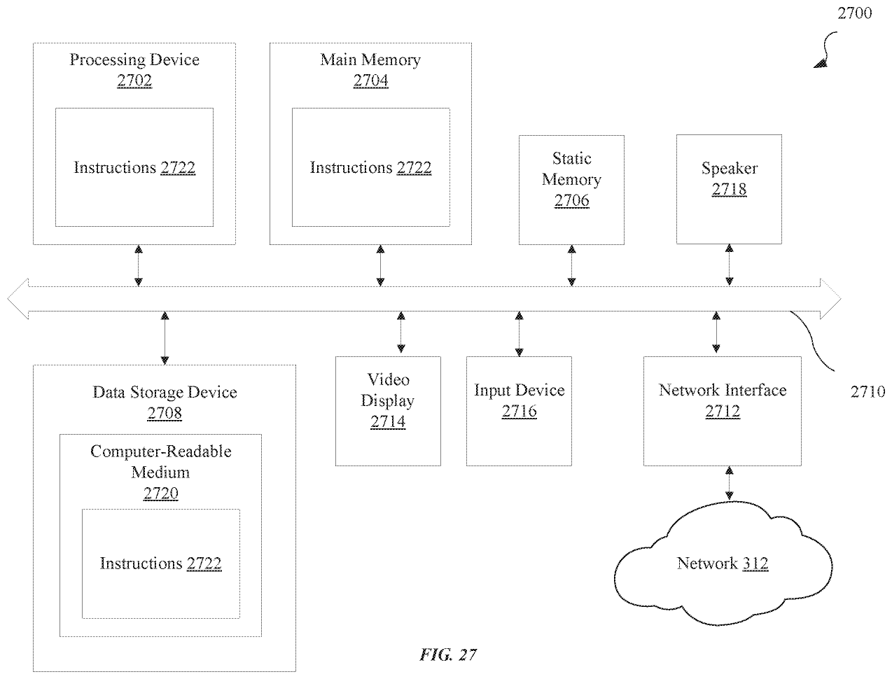

FIG. 27 shows an example computer system in accordance with at least some embodiments. In one example, computer system 2700 may correspond to any of the computing devices 324 or the central services system 300 of FIG. 3. The computer system 2700 may be capable of executing the data exchange engine 302, the analytics engine 304, the Clinician selection engine 310, the training engine 311, the patient application 324, the EMR platform 314, the user interface 316, the Clinician application 318, the Ortho Office application 322, and/or the Ortho MD/PA application of FIG. 3. The computer system 2700 may be connected (e.g., networked) to other computer systems in a LAN, an intranet, an extranet, or the Internet. The computer system 2700 may operate in the capacity of a server in a client-server network environment. The computer system 2700 may be a personal computer (PC), a tablet computer, a wearable (e.g., wristband), a set-top box (STB), a personal Digital Assistant (PDA), a mobile phone, a camera, a video camera, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 2700 includes a processing device 2702, a main memory 2704 (e.g., read-only memory (ROM), solid state drive (SSD), flash memory, dynamic random access memory (DRAM) such as synchronous DRAM (SDRAM)), a static memory 2706 (e.g., solid state drive (SSD), flash memory, static random access memory (SRAM)), and a data storage device 2708, which communicate with each other via a bus 2710.

Processing device 2702 represents one or more general-purpose processing devices such as a microprocessor, central processing unit, or the like. More particularly, the processing device 2702 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2702 may also be one or more special-purpose processing devices such as an application specific integrated circuit (ASIC), a field programmable gate array (FPGA), a digital signal processor (DSP), network processor, or the like. The processing device 2702 is configured to execute instructions for performing any of the operations and steps discussed herein.

The computer system 2700 may further include a network interface device 2712 communicatively coupled to the network 312. The computer system 2700 also may include a video display 2714 (e.g., a liquid crystal display (LCD) or a cathode ray tube (CRT)), one or more input devices 2716 (e.g., a keyboard and/or a mouse), and one or more speakers 2718 (e.g., a speaker). In one illustrative example, the video display 2714 and the input device(s) 2716 may be combined into a single component or device (e.g., an LCD touch screen).

The data storage device 2716 may include a computer-readable medium 2720 on which the instructions 2722 (e.g., implementing the patient application 324, the EMR platform 314, the user interface 316, the Clinician application 318, the Ortho Office application 322, and/or the Ortho MD/PA application executed by any device and/or component depicted in the FIGURES and described herein) embodying any one or more of the methodologies or functions described herein are stored. The instructions 2722 may also reside, completely or at least partially, within the main memory 2704 and/or within the processing device 2702 during execution thereof by the computer system 2700. As such, the main memory 2704 and the processing device 2702 also constitute computer-readable media. The instructions 2722 may further be transmitted or received over a network 312 via the network interface device 2712.

While the computer-readable storage medium 2720 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

What is claimed is:

1. A computer-implemented method comprising:
receiving a referral comprising patient data, wherein the patient data indicates a patient is referred for in-home therapy;
generating, by a processing device, a group of candidate clinicians from a dataset of clinicians, wherein the group of candidate clinicians are matches for providing the in-home therapy to the patient, and the generating is based at least on criteria comprising a candidate clinician having the certain discipline, a current utilization rate of the candidate clinician relative to an overall capacity of the candidate clinician to work over a period of time, and a billing rate the candidate clinician charges per visit; and
assigning, by the processing device, one clinician from the group of candidate clinicians to perform the in-home therapy for the patient.

2. The computer-implemented method of claim 1, further comprising:
transmitting a notification to a computing device of each candidate clinician in the group of candidate clinicians, wherein the notification indicates the candidate clinician is identified as a match to provide the in-home therapy to the patient;
receiving, from computing devices of a subset of the group of candidate clinicians, indications of interest to perform the in-home therapy for the patient; and
wherein assigning one clinician further comprises assignment of one clinician from the subset of the group of candidate clinicians to perform the in-home therapy for the patient.

3. The computer-implemented method of claim 2, wherein transmitting the notification to the computing device of each candidate clinician in the group of candidate clinicians further comprises at least one action in a group comprising: sending a text message to the computing device of each candidate clinician; sending an electronic mail message to the computing device of each candidate clinician; and sending a push notification to an application running on the computing device of each candidate clinician.

4. The computer-implemented method of claim 1, wherein assigning further comprises automatically assigning the one clinician from the group of candidate clinicians if the one clinician has indicated a preference for automatic assignments upon matching for providing the in-home therapy to patients.

5. The computer-implemented method of claim 1, wherein the criteria further comprises at least one of:
a distance to the patient from a daily starting point of the candidate clinician,
a gender of the patient, the candidate clinician, or both,
an ethnicity of the patient, the candidate clinician, or both,
a spoken language of the patient, the candidate clinician, or both,
whether the candidate clinician is trained on an electronic medical record system used by a source from which the referral was received,
whether the candidate clinician is trained in a specialty needed to treat the patient,
whether an assistant is to aid the candidate clinician in the in-home therapy, and a status of the candidate clinician.

6. The computer-implemented method of claim 1, wherein the referral comprises at least one of a request for a specific clinician to be assigned for the in-home therapy or a request for a specific clinician to be excluded from being assigned for the in-home therapy.

7. The computer-implemented method of claim 1, wherein generating the group of candidate clinicians further comprises ranking the group of candidate clinicians in an order based on the criteria.

8. The computer-implemented method of claim 7, further comprising:
generating a rating for each candidate clinician in the group of candidate clinicians;
wherein the rating is considered when ranking the group of candidate clinicians in the order.

9. The computer-implemented method of claim 1, further comprising:
adding a weight to the criteria related to the current utilization rate of the candidate clinician relative to the overall capacity of the candidate clinician to work over the period of time, wherein the weight increases a probability that the candidate clinicians who are under-utilized relative to their overall capacity to work over the period of time are included in the group of candidate clinicians.

10. The computer-implemented method of claim 1, wherein generating the group of candidate clinicians further comprises supplying a set of patient data to an artificial intelligence engine, the artificial intelligence engine accesses a database of clinician data and selects the group of candidate clinicians based on the set of patient data and the database of clinician data.

11. The computer-implemented method of claim 1, wherein assigning, by the processing device, the one clinician from the subset of the group of candidate clinicians to perform the in-home therapy for the patient further comprises:
selecting, by an artificial intelligence engine, the one clinician from the group of candidate clinicians.

12. A system comprising:
a memory storing instructions; and
a processor communicatively coupled to the memory, wherein, when the instructions are executed by the processor, the instructions cause the processor to:
receive a referral comprising patient data, wherein the patient data indicates a patient is referred for in-home therapy;
generate a group of candidate clinicians from a dataset of clinicians, wherein the group of candidate clinicians are matches for providing the in-home therapy to the patient, and the generating is based at least on criteria comprising a candidate clinician having the certain discipline, a current utilization rate of the candidate clinician relative to an overall capacity of the candidate clinician to work over a period of time, and a billing rate the candidate clinician charges per visit; and
assign one clinician from the group of candidate clinicians to perform the in-home therapy for the patient.

13. The system of claim 12, wherein, when the instructions are executed by the processor, the instructions further cause the processor to:
transmit a notification to a computing device of each candidate clinician in the group of candidate clinicians, wherein the notification indicates the candidate clinician is identified as a match to provide the in-home therapy to the patient;
receive, from computing devices of a subset of the group of candidate clinicians, indications of interest to perform the in-home therapy for the patient; and
wherein assigning one clinician further comprises assignment of one clinician from the subset of the group of candidate clinicians to perform the in-home therapy for the patient.

14. The system of claim 13, wherein transmitting the notification to the computing device of each candidate clinician in the group of candidate clinicians further comprises at least one action in a group comprising: sending a text message to the computing device of each candidate clinician; sending an electronic mail message to the computing device of each candidate clinician; and sending a push notification to an application running on the computing device of each candidate clinician.

15. The system of claim 12, wherein assigning further comprises automatically assigning the one clinician from the group of candidate clinicians if the one clinician has indicated a preference for automatic assignments upon matching for providing the in-home therapy to patients.

16. The system of claim 12, wherein the criteria further comprises at least one of:
a distance to the patient from a daily starting point of the candidate clinician,
a gender of the patient, the candidate clinician, or both,
an ethnicity of the patient, the candidate clinician, or both,
a spoken language of the patient, the candidate clinician, or both,
whether the candidate clinician is trained on an electronic medical record system used by a source from which the referral was received,
whether the candidate clinician is trained in a specialty needed to treat the patient,
whether an assistant is to aid the candidate clinician in the in-home therapy, and a status of the clinician.

17. The system of claim 12, wherein the referral comprises at least one of a list of clinicians to be considered for the in-home therapy or a list of clinicians to be excluded from consideration for the in-home therapy.

18. The system of claim 12, wherein generating the group of candidate clinicians further comprises ranking the group of candidate clinicians in an order based on the criteria in the group.

19. The system of claim 18, further comprising:
generating a rating for each candidate clinician in the group of candidate clinicians,
wherein the rating is considered when ranking the group of candidate clinicians in the order.

20. A tangible, non-transitory computer-readable medium storing instructions that, when executed by a processor, cause the processor to:
receive a referral comprising patient data, wherein the patient data indicates a patient is referred for in-home therapy;
generate a group of candidate clinicians from a dataset of clinicians, wherein the group of candidate clinicians are matches for providing the in-home therapy to the patient, and the generating is based at least on criteria comprising a candidate clinician having the certain discipline, a current utilization rate of the candidate clinician relative to an overall capacity of the candidate clinician to work over a period of time, and a billing rate the candidate clinician charges per visit; and
assign one clinician from the group of candidate clinicians to perform the in-home therapy for the patient.
